(19) United States
(12) Patent Application Publication
(10) Pub. No.: US 2008/0138391 A1
(43) Pub. Date: Jun. 12, 2008

(54) SKIN-FRIENDLY DRUG COMPLEXES FOR TRANSDERMAL ADMINISTRATION

(76) Inventors: Dario Norberto CARRARA, Oberwil (CH); Arnaud Grenier, Steinbrunn le Haut (FR); Celine Decaudin, Saint Louis (FR); Christelle Rogue, Bouxwiller (FR); Ingo Alberti, Huningue (FR); Laetitia Delpy, Saint Louis (FR)

Correspondence Address: WINSTON & STRAWN LLP;PATENT DEPARTMENT, 1700 K STREET, N.W., WASHINGTON, DC, 20006, US

(21) Appl. No.: 11/952,857

(22) Filed: Dec. 7, 2007

Related U.S. Application Data
(60) Provisional application No. 60/869,182, filed on Dec. 8, 2006.

(51) Int. Cl. A61K 9/70; A61K 31/74

(52) U.S. Cl. 424/449; 424/78.08

(57) ABSTRACT

The present invention generally relates to pharmaceutical compositions for the treatment of various diseases and disorders, in particular the use of novel complexes of amine drugs with polyacrylic acid carbomer polymers. The compositions of the present invention can be administered transdermally or transmucosally to patients in need thereof for a systemic or for a local therapeutic effect. The compositions of the present invention present the additional benefits of being free or substantially free of excipients which may potentially be responsible for skin local reactions and unpleasant smell.

This application claims the benefit of application 60/869,182 filed Dec. 8, 2006, the entire content of which is expressly incorporated herein by reference thereto.

### FIELD OF INVENTION

The present invention relates to pharmaceutical compositions which comprise a complex of a pharmaceutically active agent with an acrylic acid polymer, and to a method of producing the same.

The present invention also relates to methods of treatments comprising administering transdermally pharmaceutical compositions of the present invention to a patient in need thereof.

### BACKGROUND OF THE INVENTION

Drugs which are insoluble or only sparingly soluble in water and/or unstable in water are generally difficult to formulate into pharmaceutical preparations. Thus, they are usually made into administrable forms by such techniques as preparation of soluble derivatives, solubilization in organic solvents, emulsification, clathration, entrapping in liposomes, entrapping in cyclodextrins, microencapsulation, or the like.

Complexation is one of several ways to favorably enhance the physicochemical properties of pharmaceutical compounds. Generalities on drug complexation and complexation techniques are discussed by G. N. Kalinkova in “Complexation: Non-Cyclodextrins”, in “Encyclopedia of Pharmaceutical Technology”, Marcel Dekker, 2002, pages 559-568.

One technique of complexation consists in forming drug complex with cyclodextrins. In the context of pharmaceutical active agents, it is known that cyclodextrins may entrap pharmaceuticals to form complexes with improved stability and/or enhanced stability. See, for instance, U.S. Pat. Nos. 5,134,127, 5,376,645, and 6,133,248, and 6,951,846, the entire contents of which are incorporated herein as reference. However, cyclodextrins present a lot of drawbacks, such as a low encapsulation yields, complex encapsulation processes, a limited solubility in water and in hydro-organic solvent media, no positive effect or even negative effect on drug delivery through lipophilic membrane barriers, possible decrease of bioavailability of Class I drugs according to the FDA's biopharmaceutics classification system (BCS), and the uncertain regulatory acceptance surrounding cyclodextrin-containing drug products. See “Complexation and Cyclodextrins”, by G. Mosher and D. O. Thompson, in “Encyclopedia of Pharmaceutical Technology”, Marcel Dekker, 2002, pages 531-558.

Another technique of complexation consists in forming drug complex with ionic polymers, such as ion exchange resins. It is well known that these resins are capable of exchanging a cation or an anion for a variety of ions brought into contact with the resin. In the context of pharmaceutical active agents, it is known that ion exchange resins may be bonded to pharmaceuticals to form pharmaceutical/resin complexes having sustained release characteristics. See U.S. Pat. Nos. 2,990,332; 3,143,465; and 4,221,778; Borodkin et al., “Interaction of Amine Drugs with a Polycarboxylic Acid Ion-Exchange Resin,” J. Pharm. Sci. 59(4): 481-486 (1970); Hinsvark et al., “The Oral Bioavailability And Pharmacokinetics Of Soluble And Resin-Bound Forms Of Amphetamine And Phentermine in Man,” J. Pharmacokinetics And Biopharmaceutics 1(4): 319-328 (1973); Schlichting, “Ion Exchange Resin Salts For Oral Therapy I, Carbinoxamine,” J. Pharm. Sci. 51(2): 134-136 (1962); Smith et al., “The Development Of A Liquid Antihistaminic Preparation With Sustained Release Properties,” J. Amer. Pharm. Assoc. 49(2): 94-97 (1960); Hirscher et al., “Drug Release From Cation Exchange Resins,” J. Amer. Pharm. Assoc. NS2(2): 105-108 (1962); Amsel et al., “Dissolution And Blood Level Studies With a New Sustained Release System,” R & SDC Proceedings 3:93-106 (1980). In addition, it is known that ion exchange resins may be bound to pharmaceutical active agents in order to eliminate taste and odor problems in oral pharmaceutical dosage forms. See Borodkin et al., “Polycarboxylic Acid Ion-Exchange Resin Adsorbates for Taste Coverage in Chewable Tablets,” J. Pharm. Sci. 60(10): 1523-1527 (1971); Specification Sheets for Amberlite IRP-64, Amberlite IRP-69 and Amberlite IRP-276, published by Rohm and Haas Company (1983). In U.S. Pat. No. 5,188,825, the entire content of which is herein incorporated as reference, Iles et al., disclose freeze-dried dosage forms including a substantially water insoluble complex of a water soluble active agent bonded to an ion exchange resin. Freeze-dried dosage forms are claimed to exhibit enhanced compositional and physical stability. Active agents consist in water soluble salts having eutectic melting characteristics of phenylephrine, chlorpheniramine, triprolidine, pseudoephedrine and phenylpropanolamine, all antihistaminic drugs for relief of congestion or stuffiness in the nose caused by hay fever or other allergies, common colds, or sinus trouble. Ion exchange resins consist in gel type resins (formed from the copolymerization of styrene and divinylbenzene, such as AMBERLITE™ Resin Grade IRP-69 or AMBERLITE™ Resin Grade IRP-276) and macroreticular type resins (formed from the copolymerization of methacrylic acid and divinylbenzene, such as AMBERLITE™ Resin Grade IRP-64). Preparation of the active agent/ion exchange resin complexes involves numerous steps: after having been washed, dried and sieved, the ion exchange resin is suspended under stirring in an aqueous solution containing the active agent. The resulting active agent/resin complex is then isolated and purified by decantation/washing and then dried prior to incorporation in oral dosage forms. Most preferred weight ratios of active agents to ion exchange resins range from about 1:1 to about 1:3. Benefit of the complexation is the absence of formation of eutectic point or glass point system which would make freeze-drying technique not applicable.

However, even by such complexation procedures it is generally still difficult to obtain preparations that would allow the drug to display its action fully as will be disclosed by the present invention.

Polyacrylic acid (or carboxypolymethylene polymers, or polyacrylates, or acrylic acid polymers), are well known in the pharmaceutical, cosmetic and food industry. More particularly, these polymers are widely used in the pharmaceutical industry as dispersing, emulsifying, suspending or thickening agents. Such polymers are available from Noveon, Inc (Cleveland, Ohio, USA), under the trademarks CARBOPOL®, PEMULEN®, NOVEON® Polycarbophil. The USP-NF, European Pharmacopoeia, British Pharmacopoeia, United States Adopted Names Council (USAN), and International Nomenclature for Cosmetic Ingredients (INCI) have adopted the generic (i.e., non-proprietary) name “carbomer” for various homopolymer polymers. The Japanese Pharmaceutical Excipients list carbomer homopolymers as “carboxyvinyl polymer” and “carboxy polymethylene.” The Italian Pharmacopoeia also identifies Carbopol 934P as “carboxy polymethylene” and the Deutschen Artzneibuch calls Carbopol 980NF “polyacrylic acid.” Carbopol copolymers, such as Carbopol 1342 NF and 1382, and the PEMULEN® polymeric emulsifiers, have also been named “carbomer” by the USP-NF, but are considered “Acrylates/C10-C30 Alkyl Acrylates Crosspolymer” by the INCI. The NOVEON® series of products is generically known as “polycarbophil”. All of these polymers have the same acrylic acid backbone. The main differences are related to presence of comonomer and crosslink density. Specifically, the polymers are either homopolymers of acrylic acid cross-linked with allyl sucrose or allyl pentaerythritol (CARBOPOL® homopolymers); homopolymers of acrylic acid cross-linked with divinyl glycol (NOVEON® polycarbophils); or copolymers of acrylic acid with minor levels of long chain alkyl acrylate comonomers crosslinked with allylpentaerythritol (CARBOPOL® copolymers and PEMULEN® polymeric emulsifiers). The molecular weight of these polymers is theoretically estimated to range from 700,000 to 3 or 4 billion. All these polymers are herein designated as carbomers. In most liquid systems, carbomers require neutralization to thicken most efficiently. Sodium hydroxide, potassium hydroxide, ammonium hydroxide, and some water-soluble organic amines are excellent neutralizing agents for carbomers in water systems. In all cases the solution viscosity increases as the various carbomers are neutralized.

A flat plateau is reached for the pH range of 5 to 10 and a loss in efficiency occurs as higher pH is obtained. Apart from neutralization with bases, carbomer dispersions can also be thickened by another mechanism, called hydrogen-bonding. Some commonly used hydroxyl donors are: polyols (such as glycerin, propylene glycol and polyethylene glycols), sugar alcohols such as mannitol, nonionic surfactants with five or more ethoxy groups, glycol-silane copolymers, polyethylene oxide, and fully hydrolyzed polyvinyl alcohol, among others. These reagents hydrogen-bond with the polymer molecule causing it to uncoil: see Noveon's Technical Data Sheet 43, “CARBOPOL® polymers can thicken without neutralization”, January 2002. Amino acids are also useful as neutralizing agents for CARBOPOL® polymers: see Noveon's Technical Data Sheet 53, “Amino Acid Salts of CARBOPOL® Polymers”, January 2002. The man of the art is kindly asked to refer to Noveon's Products Specifications, Pharmaceutical Bulletins and Technical Data Sheets for further description and information on carbomer polymers of interest in the present invention.

In the context of pharmaceutical active agents, it is known that the presence of carboxy groups in carbomers makes them ionic and permits of the formation of salts such as metal salts, and other complexes. Some of these complexes have modified characteristics, as described herein after.

In U.S. Pat. Nos. 4,808,411, to Lu et al., and 5,945,405, to Spanton et al., the entire contents of which are herein incorporated as reference, authors disclose complexation of carbomer and erythromycin (or derivatives thereof), an antibiotic useful in treatment of common pediatric infections of the middle ear and upper respiratory tract, as well as certain forms of pneumonia which afflict the elderly. Complexation allows for providing acceptable palatable dry and liquid dosage forms for oral administration by masking the very bitter taste of erythromycin derivatives. Formation of erythromycin derivatives-carbomer complex involves evaporation of organic solvent and drying. The lowest ratio of active drug to carbomer is sought as this minimizes the release of free drug in water, which is critical for both stability of the drug (drug degradation occurs primarily in the aqueous phase) and palatability of the composition (significant perception of bitterness in the mouth).

In U.S. Pat. No. 5,225,189, the entire content of which is herein incorporated as reference, Pena provides a method of producing an acceptable and cosmetically elegant gel of minoxidil, an antihypertensive agent also useful to grow hair when applied topically. More particularly, U.S. Pat. No. 5,225,189 teaches how to prevent the formation and the precipitation of an undesired minoxidil-carbomer complex by adding to the carbomer dispersion a solution which comprises the neutralizing amine, namely diisopropylamine, together with the minoxidil drug.

In U.S. Pat. No. 5,843,482, the entire content of which is herein incorporated as reference, Rhodes et al., disclose pharmaceutical compositions comprising water-soluble complexes of carbomer and bismuth (a metal), or salts thereof, for the treatment of Helicobacter pylori infection and inflammatory bowel disease. Compositions are intended for oral and rectal administration. Complexes have the advantage of being very poorly absorbed from the gut, thereby limiting the absorption of bismuth in the gut, known to be responsible for unwanted side-effects which may limit the duration, dosage or intensity of bismuth treatments of the alimentary canal. Formation of complex involves, as described in Example 1, dispersion under vigorous stirring in water of bismuth and carbomer, then gradual addition of a sodium hydroxide solution of known strength, preferably 20% w/v, until a viscous solution (gel) is formed and the pH is adjusted to between 6 and 7.5, then extraction of the carbomer/bismuth complex from the aqueous solution by precipitation with organic solvent, then drying for use in dry formulations or re-solubilization for use in an enema. Preferred ratios of bismuth to carbomer are those ratios where carbomer is present in excess to solubilize the bismuth but preferably not so much that over-viscous solutions are produced.

In International Patent Application WO 97/038726, the entire content of which is herein incorporated as reference, Sachetto et al., further improved therapeutic potential of bismuth carbomer complexes by coating particles of the carbomer complex with a water insoluble anionic polymer.

In U.S. Pat. No. 5,846,983, the entire content of which is herein incorporated as reference, Sandborn et al., disclose complex of nicotine and crosslinked polyacrylic acid polymers for the treatment of inflammatory bowel disease in the form of oral or rectal dosage forms. Preparation of nicotine complexes comprises addition of an organic solution of nicotine in a colloidal dispersion of carbomer until thickening occurs, then drying, then formulation into oral solid dosage forms or re-suspended in rectal flowable dosage forms. Noteworthy, rectal drug carrier vehicles are preferably thickened by further addition of thickeners. Claimed benefit of these complexes is a delayed release and absorption of nicotine.

In U.S. Pat. No. 6,071,959, the entire content of which is herein incorporated as reference, Rhodes et al., disclose complexes of amide-type local anesthetics and carbomer effective for the treatment of pain, and in particular for the treatment of inflammatory bowel disease. Complexes are in the form of oral or rectal dosage forms.

In U.S. Pat. No. 6,238,689, the entire content of which is herein incorporated as reference, Rhodes et al., disclose complexes of nicotine and carbomer delivered for absorption from the intestine for the treatment of nicotine responsive conditions particularly schizophrenia, Alzheimer's disease, Tourette's syndrome, Parkinson's disease, depression (particularly associated with cessation of smoking), inflammatory skin conditions, and as an aid to cease smoking. Complexes are delivered as post-gastric delayed release oral dosage forms as a pill, tablet, powder or capsule, or as a flowable liquid carrier as an enema. In the case of enema, the pH is adjusted to about pH 5.0 (at which patients feel comfortable) by adding quantities of a suitable organic amine such as trometamol to the preparation, which simultaneously neutralizes some of the carbomer molecules thereby increasing the viscosity. When trometamol is used as a buffer instead of e.g. phosphate buffer, the nicotine peak plasma concentration is significantly lowered, thereby further improving the beneficial treatment of the invention since nausea and other side-effects are induced by peak plasma levels.

It is noted that carbomer has been reacted with basic drugs, such as the ones mentioned herein above, but it has not been suggested previously that the formation of basic drug-carbomer complexes modify or enhance their physical and chemical characteristics as well as their pharmacological effect when administered transdermally. More particularly, the prior art has not suggested that the complexation of basic drug with carbomers enables to significantly delay crystallization or precipitation of said basic drugs and thereby maintain drug thermodynamic activity at a high level, which is an up most prerequisite for enhanced skin drug penetration.

Administration of any active pharmaceutical agent should preferably be provided by an administration regime—the route of administration and the dose regimen—that is as simple and non-invasive as possible in order to maintain a high level of compliance by the patient. Oral administration is an administration regime that is commonly used because it is relatively simple to follow, but oral administration may cause many side effects and complications, including, among others, complications associated with gastrointestinal irritation and drug metabolism in the liver. For instance, oral administration of pramipexole can cause serious adverse effects such as nausea, dizziness, drowsiness, somnolence, insomnia, constipation, unusual weakness, stomach upset and pain, headache, dry mouth, hallucinations, difficulty moving or walking, difficulty breathing, confusion, restlessness, leg or foot swelling, fainting, twitching, chest pain, unusually fast or slow heartbeat, muscle pain, vision problems, fever, severe muscle stiffness, and sudden irresistible urge to sleep. Even administration of small amounts of pramipexole, which is typically administered at a daily does of about 1.5 to 4.5 mg, with bioavailability of 90%, is associated with considerable side effects. Oral administration of oxybutynin is also associated with common anticholinergic adverse events, e.g. dry mouth, blurred vision, constipation, drowsiness. An alternative route of administration which would alleviate side effects and would improve patient tolerance is therefore desired.

Recently, administration of pharmaceutical active agents through the skin—the “transdermal drug delivery”—has received increased attention because it provides not only a simple dosage regime but also a relatively slow and controlled release of an active agent into the body, ensuring a safe and effective administration of the active agent. Advantageously, transdermal administration can totally or partially alleviate the side effects associated with oral administration. For example, U.S. Pat. No. 7,087,241 provides compositions and methods for administering oxybutynin transdermally while minimizing the incidence and/or severity of adverse drug experiences associated with oral oxybutynin therapy. U.S. Pat. No. 5,112,842 explains that continuous transdermal delivery of pramipexole provides a number of advantages, such as sustained pramipexole blood levels, which is believed to provide a better overall side effect profile than typically associated with oral administration; absence of first-pass effect; substantial avoidance of gastrointestinal and other side effects; and improved patient acceptance.

Transdermal administration of drugs by means of a patch, also known as transdermal therapeutic system (TTS), is known since decades. Although TTS have significant advantages, it has also limitations, such as safety issues, cutaneous reactions (see Chapman M S, Perazd J E, Perry A E, Zug K A, Brown C I, “Contact leukoderma caused by buspirone patches.”, Am J Contact Dermat. 2002 March; 13(1):46-9; see also Andrea L. Musel; Erin M. Warshaw, “Cutaneous Reactions to Transdermal Therapeutic Systems”, Dermatitis. 2006; 17(3): 109-122. ©2006 American Contact Dermatitis Society), and patient-related issues. To maintain constant delivery rates throughout the duration of application, most transdermal drug delivery systems contain 20 times the drug quantity to be absorbed while worn, producing a stable concentration gradient that ensures constant delivery. Therefore patches still contain drug after removal and used patches must be discarded readily. Damages to patches may influence drug delivery and increase skin permeability and blood flow, which may lead to increased drug absorption and resultant toxicity, followed by an abrupt drop in continuous drug delivery. Application site reactions can result from exposure to the high drug concentration per square centimeter of skin, from exposure to the adhesive, or from exposure to excipients of transdermal systems. Such reactions are further emphasized by the occlusive nature of the patches, responsible for an excessive moisture saturation underneath the patches (skin is not “breathing” anymore). Site rotation may reduce the irritation associated with repeated patch application, but if the reaction is extensive or systemic, patch use should be discontinued. Discontinuation of clinical studies attributable to application sites reactions is common with transdermal patches. The Food and Drug Administration has reported safety issues related to the use of transdermal patches. These include partial removal of the backing of the patch before application (resulting in under-dosing); application of patches on oily, inflamed, broken, shaved or calloused skin areas or on open wounds; loss of adhesion and/or detachment of patches under specific conditions (showering, bathing, excessive sweating), and cutting of reservoir patches (resulting in altered release of medication and uncontrolled drug delivery). Easy detection of patches on exposed skin is also perceived as a drawback by patients who feel stigmatized. See V. W. Nitti, S. Sanders, D. R. Stakin, R. R. Dmochowski, P. K. Sand, S. McDiamid, H. Maibach, “Transdermal delivery of drugs for urologic applications: basic principles and applications”, in Urology 67: 657-664, 2006.

All the aforementioned cons are partially or totally addressed by non-occlusive, transparent, skin-friendly transdermal semi-solid gel formulations. However, non-occlusive liquid or semi-solid dosage forms face the problem of drug stability, as drug is prone to crystallize and precipitate upon evaporation of drug carrier following transdermal administration. Importance of preventing or delaying drug crystallization to ensure optimal drug skin penetration is discussed in U.S. Patent Application No. 20060153905, the entire content of which is incorporated herein as reference. This is very often achieved by the recourse to large amounts of organic solvents and co-solvents. Benefits of minimizing or avoiding use said organic solvents and co-solvents is discussed in U.S. Patent Application No. 20070048360, the entire content of which is incorporated herein as reference.

In view of the foregoing, there is a strong unmet need for skin-friendly transdermal compositions comprising active pharmaceutical agents having enhanced stability.

There is a further need for transdermal and topical compositions of active pharmaceutical drugs wherein presence of significant amounts of organic solvents is not required to maintain said drugs in a state compatible with permeation through or penetration to the skin or the mucosa surfaces.

There is another need for transdermal and topical compositions of drugs wherein crystallization of said drugs is significantly delayed or even totally prevented without having recourse to the use of high amounts of organic solvents and co-solvents.

There is yet another need for transdermal and topical compositions of drugs with improved patient compliance and being devoid of ingredients known to be potential skin irritants, e.g. alkalis and neutralizing amines such as but not limited to sodium hydroxide, diethanolamine, triethanolamine, and diisopropylamine.

It is an object of the present invention to obviate or mitigate the aforesaid disadvantages, and to address all of the aforementioned needs by providing transdermal and topical compositions containing drugs whose stability is outstandingly maintained through the formation of drug complexes with acrylic acid polymers.

No admission is made that any reference, including any patent or patent document, cited in this specification constitutes prior art. In particular, it will be understood that, unless otherwise stated, reference to any document herein does not constitute an admission that any of these documents forms part of the common general knowledge in the art in United States of America or in any other country. The discussion of the references states what their authors assert, and the applicant reserves the right to challenge the accuracy and pertinency of the documents cited herein.

### SUMMARY OF THE INVENTION

The present invention relates to a pharmaceutical composition comprising at least one amine drug and an acrylic acid carbomer polymer in the form of a complex that delays crystallization of said at least one amine drug, enhances skin penetration of said at least one amine drug, or allows for the use of no or lower amounts of solvents or pH adjusting agents; and a pharmaceutically acceptable carrier; and optionally at least one non-amine drug. The at least one amine drug uncoils the carboxyl groups of the acrylic acid polymer in the complex so that the viscosity of the composition is not inferior to the viscosity of the same composition not containing the at least one amine drug.

The invention also relates to a method of transdermal or transmucosal systemic or local administration of a pharmaceutical composition as disclosed herein to a mammal in need thereof, wherein the mammal is a human being.

The invention also relates to the use of an acrylic acid carbomer polymer to form a complex with at least one amine drug wherein the complex delays crystallization of the at least one amine drug, enhances skin penetration of the at least one amine drug, or allows for the use of no or lower amounts of solvents or pH adjusting agents. Another use according to the invention is to form a pharmaceutical composition, wherein an acrylic acid carbomer polymer forms a complex with at least one amine drug to delay crystallization of the at least one amine drug, enhance skin penetration of the at least one amine drug, or allow for the use of no or lower amounts of solvents or pH adjusting agents.

### BRIEF DESCRIPTION OF THE DRAWINGS

The features and benefits of the invention will now become more clear from a review of the following detailed description of illustrative embodiments and the accompanying drawings, wherein:

FIG. 1 is graphic representation of relative kinetic profiles of a formulation comprising a complex of pramipexole and carbomer in accordance with the present invention (Example 5) compared with a formulation comprising pramipexole wherein carbomer is replaced by cellulose as the thickening agent (Example 4).

FIG. 2 is graphic representation of drug flux profiles of a formulation comprising a complex of pramipexole and carbomer in accordance with the present invention (Example 5) compared with a formulation comprising pramipexole wherein carbomer is replaced by cellulose as the thickening agent (Example 4).

FIG. 3 is graphic representation of relative kinetic profiles of a formulation comprising a complex of ropinirole and carbomer in accordance with the present invention compared with a formulation comprising ropinirole wherein carbomer is replaced by cellulose as the thickening agent.

FIG. 4 is graphic representation of drug flux profiles of a formulation comprising a complex of ropinirole and carbomer in accordance with the present invention compared with a formulation comprising ropinirole wherein carbomer is replaced by cellulose as the thickening agent.

FIG. 5 shows the relative drug recovery profile of lidocaine after the 24 hour biodistribution in a formulation comprising a complex of lidocaine and carbomer in accordance with the present invention (Example 17) compared with formulations comprising lidocaine wherein carbomer is replaced by hydroxypropylcellulose (Examples 15 and 16) as the thickening agent.

### DEFINITION OF TERMS

It is to be understood that the terminology used herein is for the purpose of describing particular embodiments only, and is not intended to be limiting. As used in this specification, description of specific embodiments of the present invention, and any appended claims, the singular forms “a,” “an” and “the” include plural referents unless the context clearly dictates otherwise. Thus, for example, reference to “a cosolvent” includes two or more cosolvents, mixtures of cosolvents, and the like, reference to “a compound” includes one or more compounds, mixtures of compounds, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

In describing and claiming the present invention, the following terminology will be used in accordance with the definitions set out below.

The phrase “dosage form” as used herein refers to a pharmaceutical composition comprising an active agent and optionally containing inactive ingredients, e.g., pharmaceutically acceptable excipients such as suspending agents, surfactants, disintegrants, binders, diluents, lubricants, stabilizers, antioxidants, osmotic agents, colorants, plasticizers, coatings and the like, that may be used to manufacture and deliver active pharmaceutical agents.

The phrase “gel” as used herein refers to a semi-solid dosage form that contains a gelling agent in, for example, an aqueous vehicle, an organic vehicle, a mineral oil vehicle, and mixtures thereof, wherein the gelling agent imparts a three-dimensional cross-linked matrix to the vehicle. Preferred vehicles of the present inventions are aqueous and hydroalcoholic vehicle. The term “semi-solid” as used herein refers to a heterogeneous system in which one solid phase is solubilized or suspended in a second liquid phase.

The phrase “carrier” or “vehicle” as used herein refers to carrier materials (other than the pharmaceutically active ingredient) suitable for transdermal or topical administration of a pharmaceutically active ingredient. A vehicle may comprise, for example, solvents, cosolvents, permeation enhancers, pH buffering agents, antioxidants, preservatives, gelling agents, colorants, additives, film-formers, humectants, or the like, wherein components of the vehicle are nontoxic and do not interact with other components of the total composition in a deleterious manner.

The phrase “non-occlusive transdermal or topical drug delivery” as used herein refers to transdermal delivery methods or systems that do not occlude the skin or mucosal surface from contact with the atmosphere by structural means, for example, by use of a patch device, a fixed application chamber or reservoir, a backing layer (for example, a structural component of a device that provides a device with flexibility, drape, or occlusion), a tape or bandage, or the like that remains on the skin or mucosal surface for a prolonged period of time. Non-occlusive transdermal or topical drug delivery includes delivery of a drug to skin or mucosal surface using a topical medium, for example, creams, ointments, sprays, solutions, lotions, gels, and foams. Typically, non-occlusive transdermal drug delivery involves application of the drug (in a topical medium) to skin or mucosal surface, wherein the skin or mucosal surface to which the drug is applied is left open to the atmosphere.

The phrase “occlusive transdermal or topical drug delivery” as used herein refers to transdermal delivery methods or systems that occlude the skin or mucosal surface from contact with the atmosphere by structural means, for example, by use of a patch device, a fixed application chamber or reservoir, a backing layer (for example, a structural component of a device that provides a device with flexibility, drape, or occlusion), a tape or bandage, or the like that remains on the skin or mucosal surface for a prolonged period of time. Occlusive transdermal or topical drug delivery includes delivery of a drug to skin or mucosal surface using a topical medium, for example, creams, ointments, sprays, solutions, lotions, gels, and foams under occlusion. Typically, occlusive transdermal or topical drug delivery involves application of the drug (in a topical medium) to skin or mucosal surface, wherein the skin or mucosal surface to which the drug is applied is protected from the atmosphere.

The phrase “systemic” delivery, as used herein, refers to both transdermal (and “percutaneous”) and transmucosal administration, that is, delivery by passage of a drug through a skin or mucosal tissue surface and ultimately into the bloodstream.

The phrase “topical” delivery, as used herein, refers to delivery of a drug to any accessible body surface such as, e.g. for instance the skin, the nasal mucosa, the auricular mucosa, the buccal mucosa, the ocular mucosa, the pulmonary mucosa, the vaginal mucosa and rectal mucosa, as well as gastrointestinal epithelium, that is, penetration of a drug into a skin or mucosal tissue surface for local action.

The phrase “administration of active agents” as used herein can be understood to include local administration or systemic administration. For instance in case of the transdermal route, “administration of active agents” can be understood to include local penetration into the different layers of the skin or permeation through the skin into the systemic compartments.

The phrase “therapeutic agent”, “pharmaceutical agent”, “pharmacological active agent” or “active agent”, which are used interchangeably, as used herein, can be understood to include any substance or formulation or combination of substances or formulations of matter which, when administered to a human or animal subject, induces a desired pharmacologic and/or physiologic effect by local and/or systemic action.

The phrase “excipient” as used herein refers to any inert substance combined with an active agent to prepare a convenient dosage form and vehicle for delivering the active agent.

The phrase “therapeutically effective amount” as used herein refers to a nontoxic but sufficient amount of a drug, agent, or compound to provide a desired therapeutic effect.

The phrase “substantially” as used herein refers to an amount of a present ingredient, component or additive that is less than that which is necessary to impart the characteristics of the ingredient, component or additive to the composition.

The phrase “dose” and “dosage” as used herein refers to a specific amount of active or therapeutic agents for administration.

The phrase “solvent” refers herein to “volatile solvent” and “non-volatile solvents”. A volatile solvent is a solvent that changes readily from solid or liquid to a vapor, and that evaporates readily at normal temperatures and pressures. Examples of volatile solvents include, but are not limited to, ethanol, propanol, butanol, isopropanol, and/or mixtures thereof. A non-volatile solvent is a solvent that does not change readily from solid or liquid to a vapor, and that does not evaporate readily at normal temperatures and pressures. Examples of non-volatile solvents include, but are not limited to, propylene glycol, glycerin, liquid polyethylene glycols, polyoxyalkylene glycols, and/or mixtures thereof. Stanislaus, et al., (U.S. Pat. No. 4,704,406) defined “volatile solvent” as a solvent whose vapor pressure is above 35 mm Hg when skin temperature is 32° C., and a “non-volatile” solvent as a solvent whose vapor pressure is below 10 mm Hg at 32° C. skin temperature. Solvents used in the practice of the present invention are typically physiologically compatible and used at non-toxic levels.

The phrase “cosolvent” herein refers to water-miscible organic solvents that are used in liquid drug formulations to increase the solubility of poorly water-soluble substances or to enhance the chemical stability of a drug. The phrase “solvent” and “cosolvent” as used herein are totally interchangeable.

The phrase “alcohol” as used herein refers to a short-chain C₂-C₄ alcohol, for example, ethanol, propanol, butanol, isopropanol, propylene glycol, diethylene glycol mono ethyl ether, glycofurol, and/or mixtures of thereof.

The phrase “permeation enhancer” or “penetration enhancer” as used herein refers to an agent that improves the rate of transport of a pharmacologically active agent (e.g., nicotine) across the skin or mucosal surface. Typically a penetration enhancer increases the permeability of skin or mucosal tissue to a pharmacologically active agent. Penetration enhancers, for example, increase the rate at which the pharmacologically active agent permeates through skin and enters the bloodstream. Enhanced permeation effected through the use of penetration enhancers can be observed, for example, by measuring the flux of the pharmacologically active agent across animal or human skin as described in the Examples herein below. An “effective” amount of a permeation enhancer as used herein means an amount that will provide a desired increase in skin permeability to provide, for example, the desired depth of penetration of a selected compound, rate of administration of the compound, and amount of compound delivered.

The phrase “effective” or “adequate” permeation enhancer or combination as used herein means a permeation enhancer or a combination that will provide the desired increase in skin permeability and correspondingly, the desired depth of penetration, rate of administration, and amount of drug delivered.

The phrase “thermodynamic activity” of a substance means the energy form involved in skin permeation of this substance. The chemical potential of a substance is defined in thermodynamics as the partial molar free energy of the substance. The difference between the chemical potentials of a drug outside and inside the skin is the energy source for the skin permeation process.

The term “subject” as used herein refers to any warm-blooded animal, particularly including a member of the class Mammalia such as, without limitation, humans and non human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex.

Before describing the present invention in detail, it is to be understood that this invention is not limited to particular embodiments described herein, for example, particular solvent(s), antioxidant(s), cosolvent(s), penetration enhancer(s), buffering agent(s), preservative(s), and/or gelling agent(s), and the like, as use of such particulars may be selected in view of the teachings of the present specification by one of ordinary skill in the art. It is also to be understood that the terminology used herein is for the purpose of describing particular embodiments of the invention only, and is not intended to be limiting.

### DETAILED DESCRIPTION OF THE PREFERRED EMBODIMENTS

The present invention relates to pharmaceutical compositions comprising novel drug complexes, and to methods of making same. The present invention also relates to the use of said pharmaceutical compositions for the treatment of various diseases and disorders in patients in need thereof.

Inventors have surprisingly found that certain amine drugs are capable of forming a water soluble complex with acrylic acid polymers (carbomers). Unexpectedly, the bounds between the amine drug and the carbomer polymer exhibit an outstanding stability, which translates in inhibition of drug crystallization. More unexpectedly, these complexes allows for delivery of drugs in therapeutic amounts that would make these complexes useful for the treatment of various diseases and affections.

The carbomers employed in this invention are acrylic acid polymers which are commercially available from Lubrizol Advanced Materials, Inc. and other companies and which are described in the U.S. Pharmacopoeia. Carbomers are synthetic high molecular weight polymers of acrylic acid cross-linked with allylsucrose, and contain 56 to 68% carboxylic acid groups. The average equivalent weight is 76, while the molecular weight is approximately 3 million. They have the general formula:

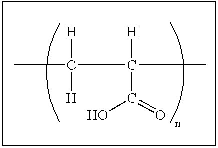

where n is from about 10,000 to about 60,000. While not intending to be limited by theory, the composition of this invention involving the combination of carbomer with a drug or its derivatives or salts may involve uncoiling of these carboxy groups. Preferred carbomers are CARBOPOL® 934, CARBOPOL® 934-P, CARBOPOL® 940, CARBOPOL® 941, CARBOPOL® 1342, CARBOPOL® 980, CARBOPOL® 981, CARBOPOL® 5984, CARBOPOL® 974P, CARBOPOL® 971P, CARBOPOL® 71G, CARBOPOL® ETD2020, CARBOPOL® ETD 2050, CARBOPOL® Ultrez 10, PEMULEN® TR1, PEMULEN® TR2, NOVEON® AA-1, NOVEON® CA 1/CA 2, all available from Lubrizol Advanced Materials, Inc., OH, USA. Polymers also suitable for the practice of the invention comprise CARBOPOL® Ultrez 20, CARBOPOL® Ultrez 21, AQUAS SF-1 and AQUAS CC, CARBOPOL® 1382, CARBOPOL 2984. In one embodiment of the present invention, the carbomer is preferably a carbomer homopolymer, such as CARBOPOL 980, or a carbomer co-polymer, such as PEMULEN® TR1 or ETD 2020.

The drugs employed in this invention to form a complex with a carbomer polymer may be any amine compound that is suitable for topical, transdermal or transmucosal delivery and induces a desired local or systemic effect. Such substances include the broad classes of compounds normally delivered through body surfaces and membranes, including skin. In general, this includes: analgesic agents; anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents; antimigraine preparations; antinauseants; antineoplastic agents; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, CNS agents; beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; ophthalmic drugs such as antiglaucoma agents; parasympatholytics; peptide drugs; psychostimulants; sedatives; steroids; sympathomimetics; tranquilizers; and vasodilators including general coronary, peripheral and cerebral. The amine drug may be one that is cosmetically or “cosmeceutically” effective rather than pharmacologically active. Such amine drugs include, for example, compounds that can reduce the appearance of aging or photodamaged skin. The amine drug may be a primary amine, a secondary amine, or a tertiary amine, or it may be an aromatic or non-aromatic nitrogen-containing heterocycle, an azo compound, an imine, or a combination of any of the foregoing. Examples of specific primary amines include, but are not limited to, amphetamine, norepinephrine, phenylpropanolamine. Examples of secondary and tertiary amines include, but are not limited to, amiodarone, amitryptyline, azithromycin, benzphetamine, bromopheniramine, chlorambucil, chloroprocaine, chloroquine, chlorpheniramine, chlorothen, chlorpromazine, cinnarizine, clarthromycin, clomiphene, cyclobenzaprine, cyclopentolate, cyclophosphamide, dacarbazine, demeclocycline, dibucaine, dicyclomine, diethylproprion, diltiazem, dimenhydrinate, diphenhydramine, diphenylpyraline, disopyramide, doxepin, doxycycline, doxylamine, dypyridame, ephedrine, epinephrine, ethylene diamine tetraacetic acid (EDTA), erythromycin, flurazepam, gentian violet, hydroxychloroquine, imipramine, isoproterenol, isothipendyl, levomethadyl, lidocaine, loxarine, mechlorethamine, melphalan, methadone, methafurylene, methapheniline, methapyrilene, methdilazine, methotimeperazine, methotrexate, metoclopramide, minocycline, naftifine, nicardipine, nicotine, nizatidine, orphenadrine, oxybutin, oxytetracycline, phenindamine, pheniramine, phenoxybenzamine, phentolamine, phenylephrine, phenyltoloxamine, procainamide, procaine, promazine, promethazine, proparacaine, propoxycaine, propoxyphene, pyrilamine, ranitidine, scopolamine, tamoxifen, terbinafine, tetracaine, tetracycline, thonzylamine, tranadol, triflupromazine, trimeprazine, trimethylbenzamide, trimipramine, trlpelennamine, troleandomycin, uracil mustard, verapamil and vonedrine. Examples of non-aromatic heterocyclic amines include, but are not limited to, alprazolam, amoxapine, arecoline, astemizole, atropine, azithromycin, benzapril, benztropine, beperiden, bupracaine, buprenorphine, buspirone, butorphanol, caffeine, capriomycin, ceftriaxone, chlorazepate, chlorcyclizine, chlordiazepoxide, chlorpromazine, chlorthiazide, ciprofloxacin, cladarabine, clemastine, clemizole, clindamycin, clofazamine, clonazepam, clonidine, clozapine, cocaine, codeine, cyclizine, cyproheptadine, dacarbzine, dactinomycin, desipramine, diazoxide, dihydroergotamine, diphenidol, diphenoxylate, dipyridamole, doxapram, ergotamine, estazolam, famciclovir, fentanyl, flavoxate, fludarabine, fluphenazine, flurazepam, fluvastin, folic acid, ganciclovir, granisetron, guanethidine, halazepam, haloperidol, homatropine, hydrocodone, hydromorphone, hydroxyzine, hyoscyamine, imipramine, itraconazole, keterolac, ketoconazole, levocarbustine, levorphone, lincomycin, lomefloxacin, loperamide, lorazepam, losartan, loxapine, mazindol, meclizine, meperidine, mepivacaine, mesoridazine, methdilazine, methenamine, methimazole, methotrimeperazine, methysergide, metronidazole, midazolam, minoxidil, mitomycin c, molindone, morphine, nafzodone, nalbuphine, naldixic acid, nalmefene, naloxone, naltrexone, naphazoline, nedocromil, nicotine, norfloxacin, ofloxacin, ondansetron, oxazepam, okycodone, oxymetazoline, oxymorphone, pemoline, pentazocine, pentostatin, pentoxyfylline, perphenazine, phentolamine, physostigmine, pilocarpine, pimozide, pramoxine, prazosin, prochlorperazine, promazine, promethazine, pyrrobutamine, quazepam, quinidine, quinine, rauwolfia alkaloids, riboflavin, rifabutin, risperidone, rocuronium, scopalamine, sufentanil, tacrine, temazepam, terazosin, terconazole, terfenadine, tetrahydrazoline, thiordazine, thiothixene, ticlodipine, timolol, tolazoline, tolazamide, tolmetin, trazodone, triazolam, triethylperazine, trifluopromazine, trihexylphenidyl, trimeprazine, trimipramine, tubocurarine, vecuronium, vidarabine, vinblastine, vincristine, vinorelbine and xylometazoline. Examples of aromatic heterocyclic amines include, but are not limited to, acetazolamide, acyclovir, adenosine phosphate, allopurinal, alprazolam, amoxapine, aminone, apraclonidine, azatadine, aztreonam, bisacodyl, bleomycin, brompheniramine, buspirone, butoconazole, carbinoxamine, cefamandole, cefazole, cefixime, cefinetazole, cefonicid, cefoperazone, cefotaxime, cefotetan, cefpodoxime, ceftriaxone, cephapirin, chloroquine, chlorpheniramine, cimetidine, cladarabine, clotrimazole, cloxacillin, didanosine, dipyridamole, doxazosin, doxylamine, econazole, enoxacin, estazolam, ethionamide, famciclovir, famotidine, fluconazole, fludarabine, folic acid, ganciclovir, hydroxychloroquine, iodoquinol, isoniazid, isothipendyl, itraconazole, ketoconazole, lamotrigine, lansoprazole, lorcetadine, losartan, mebendazole, mercaptopurine, methafurylene, methapyriline, methotrexate, metronidazole, miconazole, midazolam, minoxidil, nafzodone, naldixic acid, niacin, nicotine, nifedipine, nizatidine, omeperazole, oxaprozin, oxiconazole, papaverine, pentostatin, phenazopyridine, pheniramine, pilocarpine, piroxicam, prazosin, primaquine, pyrazinamide, pyrilamine, pyrimethamine, pyrithiamine, pyroxidine, quinidine, quinine, ribaverin, rifampin, sulfadiazine, sulfamethizole, sulfamethoxazole, sulfasalazine, sulfasoxazole, terazosin, thiabendazole, thiamine, thioguanine, thonzylamine, timolol, trazodone, triampterene, triazolam, trimethadione, trimethoprim, trimetrexate, triplenamine, tropicamide and vidarabine. Examples of azo compounds are phenazopyridine and sulfasalazine. Examples of imine compounds cefixime, cimetidine, clofazimine, clonidine, dantrolene, famotidine, furazolidone, nitrofurantoin, nitrofurazone and oxiconazole. Combinations of amine drugs and/or combinations of an amine drug with another non-amine drug may also be delivered using the methodology of the present invention. It is understood that it will appear obvious to the one skilled in the art that further active agents differing from those recited herein may fall within the scope of the present invention without significantly departing from it.

One embodiment of the invention provides a complex of a carbomer and a drug, or pharmaceutically acceptable derivative thereof, or a salt thereof. Preferably, the drug-carbomer complex is a water-soluble complex. Inventors hypothesize that the protonated amine moiety of the drug (positively charged) bounds in a non-covalent way with the anionic carboxyl groups of the carbomer (negatively charged). The inventors have surprisingly discovered that this bounding is responsible for inhibition of the crystallization of the drug upon evaporation of the drug carrier into which the drug-carbomer is embedded. Under normal condition, a liquid or semi-solid system of a solubilized drug is naturally prone to obey a thermodynamically-driven spontaneous process wherein the system tries to lower its overall energy. Drug molecules diffuse freely through the solvent system and add to the surface of another drug molecule, as molecules on the surface of a particle are energetically less stable than the ones already well ordered and packed in the interior. Drug particles, with their greater volume to surface area ratio, represent a lower energy state (and have a lower surface energy) than single molecules. Consequently, in the natural process, many small drug crystals formed initially slowly disappear, except for a few that grow larger, at the expense of the small crystals. The smaller particles continue to shrink, while larger particles continue to grow. The smaller crystals which have a higher solubility than the larger ones act indeed as fuel for the growth of bigger crystals. This phenomenon, known as Ostwald ripening, is responsible for precipitation and crystallization of drug out of liquid or semi-solid systems upon natural ageing. In the case of transdermal non-occlusive compositions, Ostwald ripening is obviously triggered by evaporation of the drug carrier upon application on the skin or the mucosa membrane. Without being bound to any theory, it is hypothesized that the physical bounding between the drug and the carbomer is responsible for keeping molecules of the drug individualized and homogeneously distributed within a three-dimensional network made of the carbomer polymer, thereby preventing initiation of the Ostwald ripening phenomenon.

In another embodiment, inventors have surprisingly discovered that drug-carbomer complexes described in the present invention provide enhanced in vitro skin permeation or penetration of drugs.

In another aspect of the present invention, patients may find compositions comprising drug-carbomer complexes of the present invention particularly comfortable and convenient to apply and to wear on the skin or the mucosa. In one embodiment, drug-carbomer complex compositions of the present invention are not tacky as similar compositions containing cellulose derivatives would be. In another embodiment, the enhanced physical stability of the drug complexed with the carbomer polymer in the composition of the present invention indeed allows for the use of no or low amounts of organic drug solvents, e.g. short-chain alcohols, which may cause skin irritation, itching, redness and dryness. In another preferred embodiment, drug-carbomer complex compositions of the present invention do not contain neutralizing base, e.g. sodium hydroxide or triethanolamine, which may also cause skin irritation, itching, redness and dryness.

According to another embodiment of the invention, there is provided a process for the preparation of a complex of a drug with a carbomer polymer wherein a drug is reacted with a polyacrylate in a liquid phase. The complex may be prepared by adding a solution of a suitable drug, e.g. the free base or a pharmaceutically acceptable salt thereof, with a colloidal dispersion of the carbomer. Alternatively, the drug may be sprinkled directly into a colloidal dispersion of the carbomer. Preferred solvent for the carbomer is pharmaceutically acceptable purified water, but non-aqueous or aqueous/organic media (e.g. alcohols or glycols or glycol ethers) can also be used if intrinsic solubilities of the reactants make it necessary. The theory governing carbomer thickening is complex and based on matching solubility parameters, hydrogen bonding and dipole moment properties of the solvent blend and the solute. If the base salt of carbomer is poorly soluble in the solvent system, thickening of carbomer will be impaired. This can even lead to precipitation of carbomer salt (phenomenon known as “salting out”), and, consequently, to absence of thickening. Therefore thorough selection of solvent media is necessary to ensure optimum thickening of carbomer salt of drugs in selected solvent system. See Noveon's Pharmaceutical Bulletin No. 8: “Noveon's polymers in semi solid products”; Noveon Pharmaceutical Bulletin No. 10: “Neutralization Procedures”; and Noveon Pharmaceutical Bulletin No. 11: “Thickening Properties”). The carbomer is added to solvent and the resulting mixture may be stirred at room temperature until a colloidal suspension forms. The dispersion may be stirred using a suitable mixer with a blade-type impeller, and the powdered carbomer slowly sieved into the vortex created by the stirrer. See Noveon's Pharmaceutical Bulletin 9 “Dispersing Procedures”. If a solvent is used for the drug, this may be a pharmaceutically acceptable organic solvent, preferably ethanol. The solution may then be added gradually to the suspension of carbomer and mixed continuously until a uniform gel has formed. A gradual thickening of the suspension may occur as neutralization of the carbomer takes place. This physical change in viscosity is consistent with neutralization of the acid by the base. In one embodiment, the resulting gel is a whitish, creamy emulgel. In a preferred embodiment, the resulting gel is transparent. Microscopic examination witness the absence of free drug suspended within the gel. The weight ratio of the drug to the carbomer for the formation of the complexes may vary greatly depending on the final pH and viscosity targeted for the gel product. Inventors have found that final pH is largely influenced by the weight ratio of the drug to the carbomer: in the case of the drug, such as the anti-Parkinson drug, is present as a free base, the higher the weight ratio of the anti-Parkinson drug to the carbomer, the higher the final pH; similarly, in the case of the drug is present as a pharmaceutically acceptable salt of a weak acid, the higher the weight ratio of the anti-Parkinson drug to the carbomer, the lower the final pH. The type of carbomer does not affect significantly the pH of the final gel product, since pH of all type of carbomer colloidal dispersions (i.e. un-neutralized) exhibit more or less the same acidic value. Besides controlling the final pH by playing on the weight ratio of the drug to the carbomer, inventors have also found the way to control final viscosity of the gel product. It is well known by the one ordinary skilled in the art that carbomer dispersions may be gradually neutralized by inorganic or organic bases until the desired degree of thickening is reached. Thus pH and viscosity are inter-depending on each other, i.e. it is not possible to increase viscosity of the carbomer dispersion while decreasing its pH. Inventors have herein surprisingly discovered that it is possible to control independently pH and viscosity of a composition of the present invention by adding the drug as a mixture of free base and a pharmaceutically salt thereof. For instance, adding the hydrochloride salt of a drug into a composition comprising a carbomer complex of the same drug as the free base would result in a decrease of the pH of said composition while viscosity of said composition would further increase. Minimal requirement for the practice of the present invention is that carbomer is present in an amount sufficient enabling solubilization of the drug. The weight ratio of reactants used of course depends on the drug used and on the proportion of free carboxyl groups in the carbomer or other carbomer. Advantageously a weight ratio of the drug to carbomer would typically be in the range 1:10 to 10:1; preferably 1:5 to 5:1, and more preferably 1:3 to 3:1. Viscosity of compositions containing such drug-carbomer complexes may be affected by changes in pH and/or ionic strength. Proper selection of the carbomer type (long-flow or short-flow rheology) as well as carbomer concentration must therefore drive the formulation of a composition according to the invention.

In another embodiment of the invention, the complex may be incorporated into a pharmaceutical composition to be administered either transdermally or transmucosally, e.g. as a composition to be applied on the skin, buccal mucosa, nasal mucosa, ocular mucosa, auricular mucosa, rectal mucosa, or vaginal mucosa. In case of transmucosal administration, the complexes of the present invention may be advantageously formulated as a bioadhesive dosage form, by the further addition of suitable bioadhesive agents. In a preferred embodiment, the bioadhesive agent is the carbomer that bounds with the drug. Preferred bioadhesive carbomer that may bound with drugs are CARBOPOL® 934-P, CARBOPOL® 974P, CARBOPOL® 971P, or NOVEON® AA-1 and NOVEON® CA 1/CA 2. Thus, according to another aspect of the invention, there is provided a pharmaceutical composition comprising a complex of the invention in association with one or more pharmaceutically acceptable carrier, diluent and/or excipient.

According to one most preferred embodiment of the present invention, the pharmaceutical composition takes the form of a non-occlusive, semi-solid formulation such as a gel or an emulgel (a thickened cream) which is systemically or topically transdermally or transmucosally administered to a skin or to a mucosa surface of a patient in need thereof. Useful compositions comprise an effective amount of the complex of the invention dissolved or dispersed in a suitable flowable carrier vehicle, such as pharmaceutically acceptable purified water. Unit doses of compositions can be administered from pre-filled sachets or tubes. Multi doses of compositions can be administered from metering dose dispensers or from tubes. The viscosity of the gel is preferably 5,000 to 50,000 centipoises and the pH is preferably 3.0 to 9.0. The ratio of drug to carbomer is preferably about 5:1 to 1:5. Dosages and dosage rate will depend on mode of application, dosages per day, size of patient etc, but typical daily doses range from 50 mg to 5000 mg. A preferred formulation for a gel would comprise, for example, a drug in a daily dose in the range 10 mg to 250 mg, preferably 25 mg to 200 mg, more preferably 25 mg to 100 mg. The formulation preferably contains 0.1 to 5.0% wt carbomer, e.g. CARBOPOL® ETD2020, more preferably 0.5 to 2.0%, in which the drug and the carbomer are present as a complex. In a particularly preferred gel, drug-carbomer (preferably CARBOPOL® ETD 2020) is present at about 2.00% w/w drug free base equivalent to 2.00% w/w carbomer. Surprisingly, the drug-carbomer complex in this composition is so that no additional thickening agents or buffers are further required. Thus a very simple, cost-effective, safe and well tolerated gel is provided in accordance with the invention.

In yet another embodiment of the invention, the composition comprising a drug as a complex with carbomer, unlike conventional transdermal or topical compositions which require the presence of alcohol for solubilization, can be substantially alcohol-free. In this aspect, inventors have surprisingly found out that it is possible to solubilize in water at least 3.00% w/w ropinirole free base (practically insoluble in water) as a result of complexation with carbomer. Accordingly, the adverse effects of including alcohol in a transdermal or transmucosal composition, namely skin irritation, redness, dryness, unpleasant smell, can be minimized or eliminated. Accordingly, the adverse effects of including large amounts of acidic compounds, e.g. concentrated hydrochloric acid, in order to solubilize drug free base in a transdermal or transmucosal composition, namely skin irritation, redness, and unpleasant smell, can be minimized or eliminated.

In yet another embodiment of the invention, advantageously the composition comprising a drug as a complex with carbomer does not require incorporation of further neutralizing base to form a medium-viscosity gel. Accordingly, the adverse effects of neutralizing bases, including unpleasant smell (particularly in the case of ammonium hydroxide or organic amines, and more particularly diisopropylamine, which exhibit a strong-fish-like odor), skin irritation, and local reactions, in a transdermal or transmucosal composition can be minimized or eliminated.

In yet another embodiment of the invention, the composition comprising a drug as a complex with carbomer provides enhanced transdermal or transmucosal permeation and/or drug flux of said active agent compared to transdermal or topical compositions not containing a drug as a complex with carbomer. In this aspect, inventors have surprisingly found out that at similar pH, a gel formulation comprising a complex of carbomer and pramipexole dihydrochloride 2.00% free base equivalent enables better skin penetration of pramipexole than a reference gel comprising cellulose derivative as the thickening agent. It is possible that the complexation of the anti-Parkinson drug with carbomer is responsible for an increased thermodynamic activity. Accordingly, the adverse effects of including further permeation enhancers in a transdermal or transmucosal composition, namely allergic reaction, skin irritation, itching, and unpleasant smell, can be minimized or eliminated.

In yet another embodiment of the invention, the composition comprising a drug as a complex with carbomer provides inhibition of crystallization of said drug as would normally occur if said drug would have been solubilized by the means of volatile solvents and/or stabilized by another thickening agent. In this aspect, inventors have surprisingly found out that a gel formulation comprising a complex of carbomer 2.00% w/w and pramipexole dihydrochloride 2.00% free base equivalent is substantially free of crystals of pramipexole after 72 hours conversely to a reference hydroxypropylcellulose gel formulation comprising pramipexole dihydrochloride 2.00% free base equivalent hydro-alcoholic wherein crystals of pramipexole were massively visible after as few as 3 hours. Accordingly, the drawbacks associated with drug crystallization in transdermal or transmucosal composition, including risk for clothing transfer and/or cross contamination and impairment of skin permeation, can be minimized or eliminated.

In yet another embodiment of the invention, the composition comprising an anti-Parkinson drug as a complex with carbomer provides stabilization of said anti-Parkinson drug. In this aspect, inventors have surprisingly found out that a gel formulation comprising a complex of carbomer 2.00% w/w and pramipexole dihydrochloride 2.00% free base equivalent is more stable than a reference hydroxypropylcellulose gel formulation after 3 months under accelerated ageing (40° C./75% R.H.). Accordingly, the adverse effects of including further stabilizers such as antioxidants or chelatants in a transdermal or transmucosal composition, namely allergic reaction, skin irritation, and itching, can be minimized or eliminated.

In yet another embodiment of the invention, the composition may further include a thickening agent or a thickening system. Exemplary thickening agents include, but are not limited to, cellulose derivatives such as ethylcellulose, hydroxypropylmethylcellulose (HPMC), ethyl-hydroxyethylcellulose (EHEC), carboxymethylcellulose (CMC), hydroxypropylcellulose (HPC), hydroxyethylcellulose (HEC), etc; natural gums such as arabic, xanthan, guar gums, alginates, etc; polyvinylpyrrolidone derivatives; polyoxyethylene polyoxypropylene copolymers, etc; others like chitosan, polyvinyl alcohols, pectins, veegum grades, and the like. Alternatively, other gelling agents or viscosants known by those skilled in the art may also be used. The gelling agent or thickener is present from about 0.1 to about 30% w/w depending on the type of polymer, as known by one skilled in the art. A preferred concentration range of the gelling agent(s), for example, hydroxypropylcellulose, is a concentration of between about 0.5 and about 5 weight percent, more preferred is a concentration of between about 1 and about 3 weight percent. One or more emulsifying agents or systems can be included in the pharmaceutically acceptable carrier in the present composition. Exemplary emulsifying agents or systems include, but are not limited to, non-ionic, cationic or anionic surfactants. One or more additional optional ingredients can be included in the pharmaceutically acceptable carrier in the present composition depending on the desired final product. Exemplary additional optional ingredients include, but are not limited to, volatile silicones (comprising, but not limited to, hexamethyldisiloxane, octamethyltrisiloxane, decamethylcyclopentasiloxane, dimethicone, silicone elastomer blends, silicone waxes, hydrophilic silicone fluids, cyclomethicone) which are commonly used in topical compositions to impart a silky “feel” can be included; one or more buffering agent, cosolvents, antioxidants, preservatives, humectants, sequestering agents, moisturizers, emollients, colorants, fragrances, flavors, film-forming agents, permeation enhancers, or any combination thereof. Various compounds for enhancing the permeability of skin, are known in the art and described in the pertinent texts and literature. Compounds that have been used to enhance skin permeability include: sulfoxides such as dimethylsulfoxide (DMSO) and decylmethylsulfoxide; ethers such as diethylene glycol monoethyl ether and diethylene glycol monomethyl ether; surfactants such as sodium laurate, sodium lauryl sulfate, cetyltrimethylammonium bromide, benzalkonium chloride, poloxamers, polysorbates and lecithin (U.S. Pat. No. 4,783,450); the 1-substituted azacycloheptan-2-ones, particularly 1-n-dodecylcyclazacycloheptan-2-one (available under the trademark AZONE™ from Nelson Research & Development Co., Irvine, Calif.; see U.S. Pat. Nos. 3,989,816, 4,316,893, 4,405,616 and 4,557,934); alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (see, e.g., U.S. Pat. No. 4,568,343); amides and other nitrogenous compounds such as urea, dimethylacetamide, dimethylformamide, 2-pyrrolidone, 1-methyl-2-pyrrolidone, ethanolamine, diethanolamine and triethanolamine; terpenes; alkanones; and organic acids, particularly salicylic acid and salicylates, citric acid and succinic acid. Percutaneous Penetration Enhancers, eds. Smith et al. (CRC Press, 1995) provides an excellent overview of the field and further background information on a number of chemical and physical enhancers.

The present topical composition is especially versatile in that it can be readily prepared in a various forms of formulations and dosage forms, including semi-solid forms with a viscosity ranging from very low (e.g., solutions, lotions) to very high (e.g., gels, creams). Thus, the present composition can be provided in any suitable form, including but not limited to, gel, ointment, lotion, suspension, solution, syrup, cream, microemulsion, and aerosol spray. Further, the composition can be deposited on a patch for application on skin or a body surface, or provided as a medicated dressing. It can also be incorporated within soft gelatin liquid capsules or tablets intended to be administered by the buccal route. Thus, the present invention provides an enhanced delivery of an active pharmaceutical agent in any variety of forms.

In yet another embodiment of the invention, a method for preparing a composition for enhanced transdermal or transmucosal delivery of a drug is provided. The method comprises forming a complex which includes a drug and a carbomer; and associating said mixture with a pharmaceutically acceptable carrier, such that the composition provides enhanced transdermal or transmucosal permeation of the drug.

In yet another embodiment of the invention, the method can include at least two pharmacologically active agents. Advantageously, the at least two active agents are contained within a single common composition. However, the at least two active agents can be contained in two distinct compositions, which can then be dispensed from a single common dispenser either simultaneously or consecutively. In this manner, the dispenser preferably includes at least two separate compartments in which each active agent is maintained in the dispenser separately from the other active agent. The dispenser can have a single actuator for dispensing each of the at least two active agents. Alternatively, the dispenser can have a plurality of actuators for each compartment. If desired, the at least two active agents can remain separated until dispensing. A variety of different types of dispensers can be used. For example, the dispenser can be a metered dose pump, or a dispensing tube. According to a yet further embodiment of the invention, there is provided the use of a complex of the invention in the preparation of a pharmaceutical composition for the treatment of a disease or a condition. According to a yet further aspect of the invention, there is provided a method of treating a disease or a condition which comprises the step of administering a pharmaceutically effective amount of a complex of a drug and a carbomer in a delayed or sustained-release dosage form. The pharmaceutical composition may be transdermally administered or may take the form of a delayed-release transmucosal composition.

These and other embodiments of the present invention will readily occur to those of ordinary skill in the art in view of the disclosure herein.

### EXAMPLES

The following examples are put forth so as to provide those of ordinary skill in the art with a complete disclosure and description of how to make and use the formulations, methods, and devices of the present invention, and are not intended to limit the scope of what the inventors regard as the invention. Efforts have been made to ensure accuracy with respect to numbers used (e.g., weights, temperature, volumes, etc.) but some experimental errors and deviations should be accounted for. Unless indicated otherwise, parts are parts by weight, molecular weight is weight average molecular weight, temperature is in degrees Centigrade, and pressure is at or near atmospheric.

The compositions produced according to the present invention meet the strict specifications for content and purity required of pharmaceutical products.

The in vitro human cadaver skin model has proven to be a valuable tool for the study of percutaneous absorption and the determination of topically applied drugs. The model uses human cadaver skin mounted in specially designed diffusion cells that allow the skin to be maintained at a temperature and humidity that match typical in vivo conditions (Franz, T. J., “Percutaneous absorption: on the relevance of in vitro data,” J. Invest Dermatol 64:190-195 (1975)). A finite dose (for example: 4-7 mg/cm²) of formulation is applied to the outer surface of the skin and drug absorption is measured by monitoring its rate of appearance in the receptor solution bathing the inner surface of the skin. Data defining total absorption, rate of absorption, as well as skin content can be accurately determined in this model. The method has historic precedent for accurately predicting in vivo percutaneous absorption kinetics (Franz, T. J., “The finite dose technique as a valid in vitro model for the study of percutaneous absorption in man,” In: Skin: Drug Application and Evaluation of Environmental Hazards, Current Problems in Dermatology, vol. 7, G. Simon, Z. Paster, M Klingberg, M. Kaye (Eds), Basel, Switzerland, S. Karger, pages 58-68 (1978)).

Pig skin has been found to have similar morphological and functional characteristics as human skin (Simon, G. A., et al., “The pig as an experimental animal model of percutaneous permeation in man,” Skin Pharmacol. Appl. Skin Physiol. 13(5):229-34 (2000)), as well as close permeability character to human skin (Andega, S., et al., “Comparison of the effect of fatty alcohols on the permeation of melatonin between porcine and human skin,” J. Control Release 77(1-2): 17-25 (2001); Singh, S., et al., “In vitro permeability and binding of hydrocarbons in pig ear and human abdominal skin,” Drug Chem. Toxicol. 25(1):83-92 (2002); Schmook, F. P., et al., “Comparison of human skin or epidermis models with human and animal skin in in vitro percutaneous absorption,” Int. J. Pharm. 215(1-2): 51-6 (2001)). Accordingly, pig skin may be used for preliminary development studies and human skin used for final permeation studies. Pig skin can be prepared essentially as described below for human skin.

(i) Skin Preparation.

Percutaneous absorption was measured using the in vitro cadaver skin finite dose technique. Cryo-preserved, human cadaver trunk skin was obtained from a skin bank and stored in water-impermeable plastic bags at <−70° C. until used.

Prior to the experiment, skin was removed from the bag, placed in approximately 37° C. water for five minutes, and then cut into sections large enough to fit on 1 cm² Franz Cells (Crown Glass Co., Somerville, N.J.). Briefly, skin samples were prepared as follows. A small volume of phosphate buffered saline (PBS) was used to cover the bottom of the Petri dishes. Skin disks generally depleted of fat layers were placed in the Petri dishes for hydration. A Stadie-Riggs manual tissue microtome was used for slicing excised skin samples. Approximately 2 mL of PBS was placed into the middle cavity of the microtome as slicing lubricant. Skin disks were placed, dermal side up, into the middle cavity of the microtome. Filter paper was soaked with PBS, inserted in the cavity just above the skin disk. The filter paper prevented the dermis from sliding onto the top of the cutting block and helped to insure more precise cutting. When all three blades of the microtome were assembled, the microtome was turned into the upright position. Using a regular and careful sawing motion the skin tissue was sliced in cross-section. The skin tissue slice was removed with the tweezers and placed in the Petri dish for hydration. Each skin slice was wrapped in PARAFILM® (Pechiney Plastic Packaging, Inc., Chicago, Ill.) laboratory film and placed in water-impermeable plastic bags. Skin samples were identified by the donor and the provider code. If further storage was necessary, the skin slices were stored in the freezer at −20° C. until further use.

The epidermal cell (chimney) was left open to ambient laboratory conditions. The dermal cell was filled with receptor solution. Receptor solution for in vitro skin permeations was typically an isotonic saline at physiological pH. The receptor solution may also contain a drug solubilizer, for example, to increase lipophilic drug solubility in the receptor phase. The receptor solution was typically a phosphate buffered saline at approximately pH 7.4 (PBS, pH 7.4; European Pharmacopeia, 3rd Edition, Suppl. 1999, p. 192, No. 4005000) with addition of 2% Volpo N20 (oleyl ether of polyethylene glycol—a nonionic surfactant with HLB 15.5 obtained by ethoxylation (20 moles) of oleyl alcohol (C18:1)). This solubilizer is currently used for in vitro skin permeations and is known not to affect skin permeability (Bronaugh R. L., “Determination of percutaneous absorption by in vitro techniques,” in: Bronaugh R. L., Maibach H. I. (Eds.), “Percutaneous absorption,” Dekker, New York (1985); Brain K. R., Walters K. A., Watkinson A. C., Investigation of skin permeation in vitro, in: Roberts M. S., Walters K. A. (Eds.), Dermal absorption and toxicity assessment, Dekker, New York (1998)).

All cells were mounted in a diffusion apparatus in which the dermal bathing solution (i.e., the receptor solution) was stirred magnetically at approximately 600 RPM and skin surface temperature maintained at 33.0°±1.0° C.

Integrity of each skin section was determined before application of the test products by measurement of trans-epidermal water loss (TEWL), using a TM 210 Tewameter (Courage-Khazaka, Germany). Differences between skin sections were determined statistically using unpaired p-test.

(ii) Dosing and Sample Collection.

(a) Franz Cell.

Just prior to dosing with the formulations described herein, the chimney was removed from the Franz Cell to allow full access to the epidermal surface of the skin. The formulations were typically applied to the skin section using a positive displacement pipette set to deliver approximately 6.25 uL (6.25 uL/1 cm²). The dose was spread throughout the surface with the TEFLON® (E. I. Du Pont De Nemours And Company Corporation, Wilmington Del.) tip of the pipette. Five to ten minutes after application the chimney portion of the Franz Cell was replaced. Experiments were performed under non-occlusive conditions. Spare cells were not dosed, but sampled, to evaluate for interfering substances during the analytical analysis.

At pre-selected time intervals after test formulation application (e.g., 2, 4, 8, 12, 16, and 24 h) the receptor solution was removed in its entirety replaced with fresh solution (0.1× Phosphate Buffered Saline with Volpo (Croda, Inc., Parsippany, N.J.), and an aliquot taken for analysis. Prior to administration of the topical test formulations to the skin section, the receptor solution was replaced with a fresh solution of Volpo-PBS. (Volpo (Oleth-20) is a non-ionic surfactant known to increase the aqueous solubility of poorly water-soluble compounds. Volpo in the receptor solution insured diffusion sink conditions during percutaneous absorption, and is known not to affect the barrier properties of the test skin.)

Skin samples from three cadaver skin donors were prepared and mounted onto cells. Typically, each formulation was tested in 4 replicates (3 different donors).

Each formulation was applied, typically, to triplicate sections for each donor. The receptor solution samples were typically collected at 2, 4, 8, 12, 16, and 24 hours after dosing. The receptor solution used was 1:10 PBS+0.1% Volpo. Differences between formulations were evaluated for statistical differences using standard statistical analysis, for example, the Student's t-Test.

After the last sample was collected, the surface was washed twice (0.5 mL volumes) with 50:50 ethanol:water twice to collect un-absorbed formulation from the surface of the skin. Following the wash, the skin was removed from the chamber, split into epidermis and dermis, and each extracted overnight in 50:50 ethanol:water for 24 hours prior to further analysis.

(b) Automatic Sampling

Automatic sampling was carried out essentially as described under “(a) Franz cell” above, with the exception that multiple cells were used coupled with an automatic sampling system. Skin from a single donor was cut into multiple smaller sections (e.g., punched skin disks cut to approximately 34 mm diameter) large enough to fit on 1.0 cm² Franz diffusion cells (Crown Glass Co., Somerville, N.J.). Skin thickness was typically between 330 and 700 um, with a mean of 523 um (+19.5%).

Each dermal chamber was filled to capacity with a receptor solution (e.g., phosphate-buffered isotonic saline (PBS), pH 7.4±0.1, plus 2% Volpo), and the epidermal chamber was left open to ambient laboratory environment. The cells were then placed in a diffusion apparatus in which the dermal receptor solution was stirred magnetically at ˜600 RPM and its temperature maintained to achieve a skin surface temperature of 32.0±1.0° C.

Typically, a single formulation was dosed to 2-3 chambers (comprising the same donor skin) at a target dose of about 5 uL/1.0 cm² using a calibrated positive displacement pipette. At pre-selected times after dosing, (e.g., 2, 4, 8, 12, 16, and 24 h) the receptor solution was sampled and a predetermined volume aliquot saved for subsequent analysis. Sampling was performed using a Microette autosampler (Hanson Research, Chatsworth, Calif.).

Following the last receptor solution sample, the surface was washed and the skin collected for analysis as described herein.

(iii) Analytical Quantification Methods.

Quantification of active agents was by High Performance Liquid Chromatography (HPLC) with Diode-Array and Mass spectrometry detector (HPLC/MS). Briefly, HPLC was conducted on a HEWLETT-PACKARD® (Hewlett-Packard Company, Palo Alto, Calif.) 1100 Series system with diode-array UV detector with MS detector. Appropriate solvent systems were run through appropriate columns at an appropriate flow rate. Samples were injected. Peak areas were quantified to concentration using an external standard curve prepared from the neat standard.

(iv) Data Analysis.

The permeation studies and the biodistribution studies (or mass balance studies) described herein provide data to obtain different profiles of the transdermal absorption of drugs through the skin as a function of time.

The absolute kinetic profile shows the mean cumulated drug permeated amount (e.g., μg/cm²) as a function of time (e.g., hours) and thus provides an evaluation of the daily absorbed dose (amount of drug transdermally absorbed after 24 hours of permeation).

The relative kinetic profile shows the mean cumulated drug permeated amount (e.g., percent) as a function of time (e.g., hours) and thus allows an evaluation of the percentage of the applied drug that is transdermally absorbed after a given time.

The flux profile shows the mean drug instant flux [e.g., μg/cm²/h] as a function of time (e.g., hours) and provides a time the steady-state flux is reached. This profile also provides an evaluation of the value of this steady-state flux. This value corresponds to the mean flux obtained at steady-state.

The mass balance profile shows distribution of the active compound (e.g., percent) within the different compartments as a function of time (e.g., hours), and more particularly within the stratum corneum, the epidermis, the dermis, the receptor compartment.

These different profiles provide means to evaluate, characterize, and compare formulations, as well as to assess the pharmaceutical efficacy of formulations and consequently, to optimize prototype formulations.

Following here is an exemplary description of the manufacturing process used to make the pharmaceutical compositions of the present invention. Generally, the active agent is introduced either alone or as a solution in a colloidal dispersion of carbomer. The resulting drug suspension was then homogenized under mechanical stirring (marine propeller) until complete solubilization of the active agent, witnessed by the formation of a homogeneous gel. If desired, further ingredients such as cosolvents, buffering agents, antioxidants, preservatives, permeation enhancers, etc, as mentioned herein above were added under mechanical stirring.

### Carbomer Complexes of Anti-Parkinson Drugs.

### Example 1a

Preparation of a carbomer gel of pramipexole according to the manufacturing process described in U.S. Pat. No. 5,225,189 was attempted.

| Part I | | |
|---|---|---|
| Purified water | q.s. 10.0 | g |
| Carbopol ® ETD2020 | 0.045 | g |
| Part II | | |
| Pramipexole dihydrochloride monohydrate | 0.287 | g |
| Propylene glycol | 2.000 | g |
| Ethanol | 1.900 | g |
| Diisopropylamine | 0.045 | g |
| Part III | | |
| Ethanol | 2.700 | g |

In each part, the component parts are prepared separately. Part III is then mixed with Part I. When a uniform mixture is obtained, Part II is then added under stirring. This leads to precipitation of white particles (“salting out”). Furthermore, the gel presents a typical ammonia smell. Noteworthy, diisopropylamine already exceeds at this concentration the maximum amount (0.20% w/w) referenced in FDA Inactive Ingredient Guide for topical/transdermal route.

### Example 1b

Example 1a was repeated increasing the amount of diisopropylamine up to 0.150 g (1.5% w/w), i.e. the upper limit of the range of concentration recommended in U.S. Pat. No. 5,225,189. This leads also to precipitation of white particles (“salting out”). The fishy ammonia smell is now very strong and totally unacceptable.

### Example 1c

Example 1a was repeated further increasing the amount of diisopropylamine up to 0.450 g (4.5% w/w). A very flowable semi-solid formulation (about 2,000 cP, BROOKFIELD RV-DVII+featured with a small sample adapter, spindle S29, 20 rpm, 25° C.). Though, “gel” is still opalescent, and the very high amount of diisopropylamine employed in this example makes this “gel” totally unacceptable from an aesthetic aspect (strong smell, high risk for skin irritation).

### Example 2

0.287 g of pramipexole dihydrochloride monohydrate (equivalent to 0.200 g of pramipexole free base) is sprinkled under gentle stirring over 9.713 g of a colloidal dispersion of carbomer Carbopol® ETD 2020 2.00% w/w in ethanol:purified water 50:50. A clear, homogeneous firm gel having a viscosity of about 10,000 cP (BROOKFIELD RV-DVII+ featured with a small sample adapter, spindle S29, 20 rpm, 25° C.) is obtained. Further addition of a single drop of a neutralizing base, either an inorganic base (sodium hydroxide) or an organic base (triethanolamine or diisopropylamine) lead to breakdown of the gel into a two-phase liquid system presenting an heterogeneous white precipitate (“salting out”).

Inventors have therefore found out a surprising way to manufacture a carbomer gel of pramipexole which is satisfactory from an aesthetic standpoint. The manufacturing process herein employed by inventors (absence of neutralization of carbomer by organic amines) is noteworthy against the teaching of the prior art.

### Example 3

A pH-native solution of 2.00% w/w free base equivalent of pramipexole dihydrochloride monohydrate in ethanol (50.0% w/w) and purified water (qs 100% w/w) exhibits rapidly a strong coloration (from yellowish to orangeish, then brownish) within less than one month at ambient temperature. Coloration is accelerated at higher storage temperature since coloration was already visibly detectable after as few as two days at 60° C. Unexpectedly, gel formulation of Example 2 herein above was colorless after several weeks at ambient temperature.

### Example 4

2.87 g of pramipexole dihydrochloride monohydrate (equivalent to 2 g of pramipexole free base) is dissolved in 40 g of ethanol, myristyl alcohol 1 g, 5 g of TRANSCUTOL P, and 20 g of propylene glycol (Part I). Separately a colloidal dispersion of 1.50 g of hydroxypropylcellulose in of purified water (qs 100 g) is prepared (Part II). Part I is then added drop wise into Part II under gentle mechanical stirring (marine propeller). A clear gel is obtained. Native apparent pH is about 3.0. Final viscosity is about 10,500 cP.

Gel is filled into a 15 ml aluminum laminated tube and stored at 40° C. (75% R.H.). Summary table herein below does present stability data of gel of Example 4 after 3-month storage (percentages are expressed as percent weight by weight % w/w).

| | | | | | | Impurity |
|---|---|---|---|---|---|---|
| | | Color* | | Assay of | | profile** |
| | | after 3 | Assay of | active after | Impurity | after 3 |
| | Color* at | months at | active at | 3 months at | profile** at | months at |
| | release | 40° C. | release | 40° C. | release | 40° C. |
| Example 4 | Colorless | As colored | 101.7% | 98.7% | 1 impurity | 1 impurity |
| | | as B2 | (RSD | (RSD | Sum = 0.7% | Sum = 0.5% |
| | | | 0.4%) | 2.5%) | | |

*Color is assessed according to the test of the European Pharmacopoeia, “2.2.2. Degree of coloration of liquids”, Method II, 5^{th} Edition, 2005, page 24-26. Coloration is judged unacceptable when it exceeds the coloration of the most strongly colored reference solution, i.e. reference solution 1 (e.g. when colored is ranked “>Y1” for yellow coloration, “>B1” for brownish coloration, etc . . . )
**Impurity reporting threshold: >0.1% w/w

### Example 5

2.87 g of pramipexole dihydrochloride monohydrate (equivalent to 2 g of pramipexole free base) is dissolved in 40 g of ethanol, myristyl alcohol 1 g, 5 g of TRANSCUTOL P, and 20 g of propylene glycol (Part I). Separately a colloidal dispersion of 2 g of carbomer ETD 2020 in of purified water (qs 100 g) is prepared (Part II). Part I is then added drop wise into Part II under gentle mechanical stirring (marine propeller). A clear, homogeneous firm gel having a viscosity of about 11,300 cP (BROOKFIELD RV-DVII+ featured with a small sample adapter, spindle S29, 20 rpm, 25° C.) and a pH of about 3.0 is obtained, i.e. values similar to those obtained for viscosity and pH obtained for the gel of Example 4 herein before.

Gel is filled into a 15 ml aluminum laminated tube and stored at 40° C. (75% R.H.). Summary table herein below does present stability data of gel of Example 5 after 3-month storage (percentages are expressed as percent weight by weight % w/w).

| | | | | | | Impurity |
|---|---|---|---|---|---|---|
| | | Color* | | Assay of | | profile** |
| | | after 3 | Assay of | active after | Impurity | after 3 |
| | Color* at | months at | active at | 3 months at | profile** at | months at |
| | release | 40° C. | release | 40° C. | release | 40° C. |
| Example 5 | Colorless | As | 101.7% | 99.3% | 1 impurity | 1 impurity |
| | | colored as | (RSD | (RSD | Sum = 0.1% | Sum = 0.2% |
| | | BY3 | 0.3%) | 0.1%) | | |

*Color is assessed according to the test of the European Pharmacopoeia, “2.2.2. Degree of coloration of liquids”, Method II, 5^{th} Edition, 2005, page 24-26. Coloration is judged unacceptable when it exceeds the coloration of the most strongly colored reference solution, i.e. reference solution 1 (e.g. when colored is ranked “>Y1” for yellow coloration, “>B1” for brownish coloration, etc . . . )
**Impurity reporting threshold: >0.1% w/w

Noteworthy, gel of Example 5 is less degraded physically wise (least color formation) and chemically wise (lower loss of active, lower sum of impurities) than the gel of Example 4. Inventors surmise that degradation of pramipexole involves the propylamino secondary amine group. Since thickening of carbomer by complexation with pramipexole is also involving this propylamino group, inventors surmise that the mechanism of stabilization of pramipexole is caused by the bounding of said propylamino group of pramipexole with the carboxy groups of the carbomer complex.

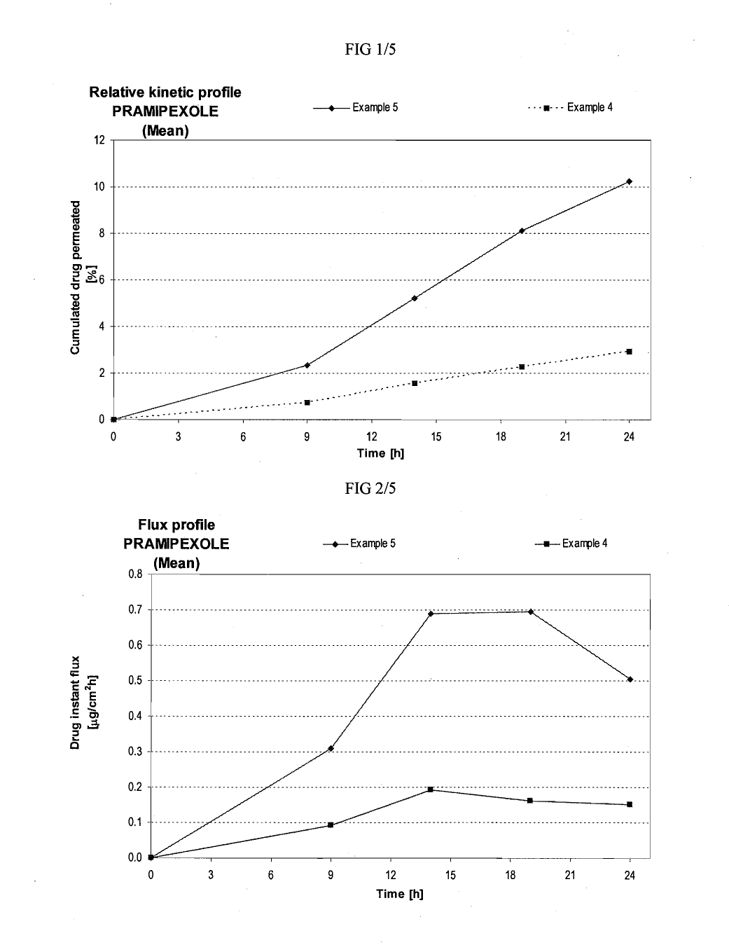

Gel compositions of Example 4 and Example 5 were then compared for in vitro skin permeation over 24 hours. The absolute kinetic delivery profile of pramipexole over the 24 hour permeation is presented in FIG. 1. In FIG. 1, the vertical axis is Cumulated Drug Permeated (μg/cm²), the horizontal axis is Time (in hours). Further, the flux results of the permeation analysis are presented FIG. 2. In FIG. 2, the vertical axis is Flux (μg/cm2/hr), the horizontal axis corresponds to sampling times (in hours). Comparison between gel composition of Example 5 (pramipexole-carbomer complex, at native pH) and Example 4 (hydroxypropylcellulose gel, at native pH) demonstrates that at similar pH, pramipexole permeates about 3.5 times more through the skin when released from the carbomer complex than from the cellulose gel. Inventors surmise that this surprising effect is caused by an increase in thermodynamic activity of pramipexole within the carbomer complex. Inventors also surmise that this may be related to inhibition of crystallization: after 3-hour exposition on a glass plate, it is observed that pramipexole begins to crystallize after, and massively crystallizes after 6 hours within the cellulose gel of Example 4, albeit carbomer gel of Example 5 shows only a very few drug crystals after 72 hours under the exact same exposure conditions.

### Example 6

0.100 g of ropinirole free base is directly sprinkled under gentle stirring over a colloidal aqueous dispersion of hydroxypropylcellulose consisting in 0.1 g of carbomer KLUCEL® HF and 9.8 g of purified water. Macroscopic examination reveals abundant free solid drug particles suspended within the gel carrier, thereby demonstrating that ropinirole free base is not solubilized.

### Example 7

0.100 g of ropinirole free base is directly sprinkled under gentle stirring over a colloidal aqueous dispersion of hydroxypropylcellulose consisting in 0.1 g of carbomer KLUCEL® HF and 9.8 g of purified water. A solution of hydrochloric acid 1M is added drop wise until complete solubilization of ropinirole free base. This requires 4.6% w/w of HCL 1M. Resulting pH is then about 3.0.

### Example 8

0.100 g of ropinirole free base is directly sprinkled under gentle stirring over a colloidal aqueous dispersion of carbomer consisting in 0.1 g of carbomer Carbopol® 980 and 9.8 g of purified water. Surprisingly and unexpectedly, a whitish, creamy, firm emulgel having a pH of 5.2 and having a viscosity of about 36,000 cP (BROOKFIELD RV-DVII+ featured with a small sample adapter, spindle S29, 20 rpm, 25° C.) spontaneously formed as a result of neutralization of carbomer by ropinirole free base. Microscopic examination revealed absence of free ropinirole particles suspended within the gel carrier at the time of manufacture. Further microscopic examination evidenced the absence of ropinirole crystals even after evaporation of the solvent system. Inventors have therefore found out a surprising way to enhance solubilization of a poorly water-soluble drug, e.g. ropinirole free base, at a pH (about 5.0) at which it would not be soluble otherwise. Further, solubilization of the non water-soluble drug is achieved without the need for adding large amount of very concentrated acids which may be present a significant potential for skin irritation and local reactions.

### Example 9

Compositions of Example 8 were prepared, varying the drug to carbomer ratio or varying the carbomer type. See Table herein below.

| | Example | | | | | |
|---|---|---|---|---|---|---|
| | 9.1 | 9.2 | 9.3 | 9.4 | 9.5 | 9.6 |
| Ropinirole form | Free | Free | Free | Free | Free | HCl |
| | base | base | base | base | base | salt |
| Ropinirole % wt | 1.0 | 2.0 | 2.0 | 1.0 | 3.0 | 3.0 FBE* |
| Carbopol ® type | 980 | 980 | 980 | 971 | ETD 2020 | ETD 2020 |
| Carbopol ® % wt | 1.0 | 0.5 | 1.0 | 1.0 | 3.0 | 3.0 |
| Vehicle | Water qs 100 | | | | Ethanol 45 | |
| | | | | | TRANSCUTOL 5 | |
| | | | | | Propylene glycol 20 | |
| | | | | | Myristyl alcohol 1 | |
| | | | | | Water qs 100 | |
| pH | 5.2 | 8.8 | 6.6 | 5.2 | 2.5 | 7.2 |
| Viscosity | 36,600 | 30,800 | 46,600 | 10,650 | 21,800 | 18,200 |

*FBE: Free Base Equivalent

As evidenced by the pH and viscosity values presented herein above, it is possible to control independently pH and viscosity of pharmaceutical compositions containing drug-carbomer complexes. For instance, pH of pharmaceutical compositions containing drug-carbomer complexes can be controlled by changing the drug to carbomer ratio or by selecting an appropriate drug form. Viscosity of pharmaceutical compositions containing drug-carbomer complexes can be controlled by selecting an appropriate carbomer type.

Noteworthy, it is also possible to modify the composition of the gel carrier in which the drug-carbomer complex is dissolved.

### Example 10

Ropinirole free base (3.00% w/w) is added into a hydro-alcoholic colloidal dispersion of TRANSCUTOL® P (5% w/w), propylene glycol (20% w/w), Carbopol® ETD 2020 (1.00% w/w), and purified water qs. A firm, homogeneous, transparent gel with a pH of about 7.8 is formed.

### Example 11

Ropinirole free base (3.00% w/w) is added into a hydro-alcoholic solution consisting in TRANSCUTOL® P (5% w/w), propylene glycol (20% w/w), and purified water qs. Ropinirole free base does not solubilize. Further addition of Carbopol® ETD 2020 (1.00% w/w) to the ropinirole free base suspension results in “salting out” of a white, flaky drug precipitate.

### Example 12

A composition of 3.00% w/w ropinirole free base in a hydro-organic media consisting of ethanol (45.0% w/w), TRANSCUTOL® P (5% w/w), propylene glycol (20% w/w), antioxidant (0.40% w/w), hydroxypropylcellulose (1.50% w/w), and purified water qs. pH is adjusted to about 7.9 by the means of hydrochloric acid 1M (5.6% w/w). Viscosity is about 10,000 cP. Presence of ethanol is herein mandatory to solubilize ropinirole free base, which would not be soluble otherwise.

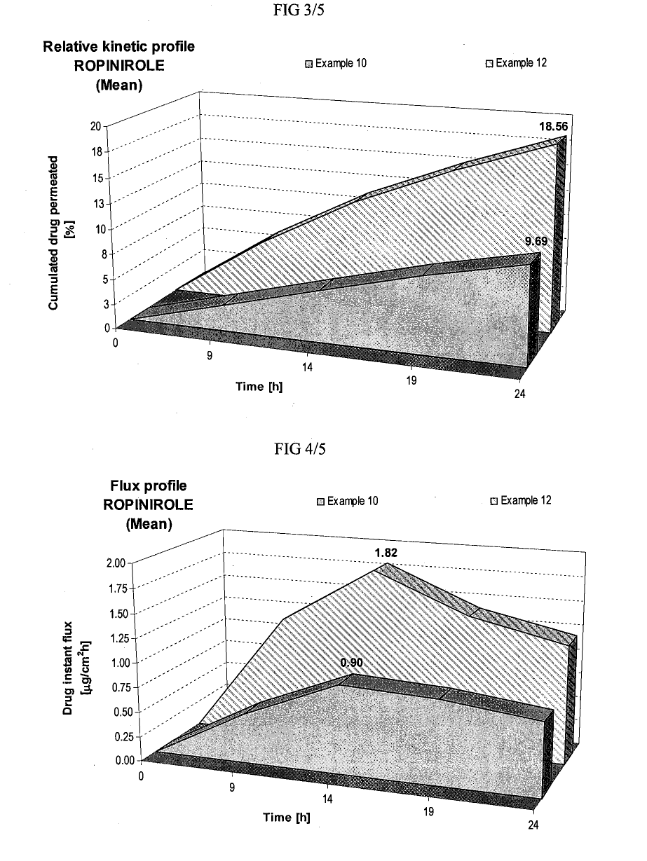

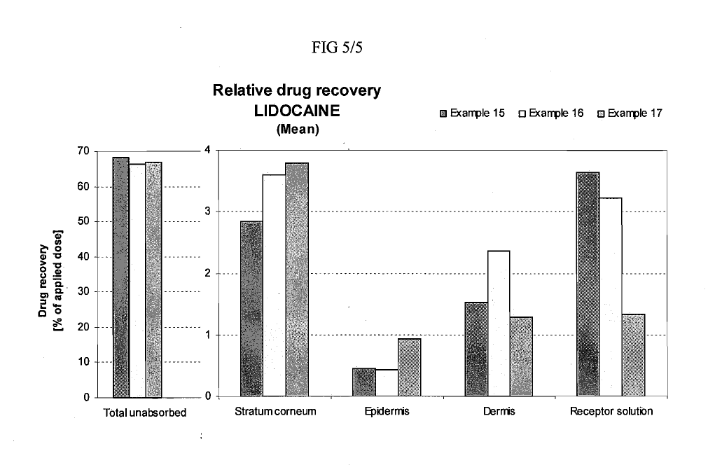

This gel is then compared to the gel composition of Example 10 for in vitro permeation of ropinirole through the skin after 24 hours. The absolute kinetic delivery profile of ropinirole over the 24 hour permeation is presented in FIG. 3. In FIG. 3, the vertical axis is Cumulated Drug Permeated (μg/cm²), the horizontal axis is Time (in hours). Further, the flux results of the permeation analysis are presented FIG. 4. In FIG. 4, the vertical axis is Flux (μg/cm2/hr), the horizontal axis corresponds to sampling times (in hours).

Comparing the hydroalcoholic gel composition of Example 12 against the aqueous gel composition of Example 10 containing the carbomer complex of ropinirole free base, one can notice that in vitro bioavailability of gel composition of Example 10 is about half of gel composition of Example 12, despite the absence of ethanol, a solvent known to be a very efficient skin permeation enhancer by fluidifying the lipids of the stratum corneum, thereby facilitating the passage of drugs.

Therefore, inventors have surprisingly found out not only a patient-friendly way to outstandingly solubilize ropinirole in semi-solid alcohol-free vehicles, but also a way to enable skin permeation of ropinirole at levels which would be sufficient for achieving therapeutic concentrations suitable for the treatment of ropinirole-responsive disease, e.g. Parkinson's Disease.

### Carbomer Complexes of Local Anesthetic Drugs.

### Example 13

Lidocaine free base (2.50% w/w) was added to an aqueous gel of hydroxypropylcellulose (1.00% w/w). A heterogeneous suspension of pH 9.43 is obtained.

### Example 14

Lidocaine free base (2.50% w/w) was added to an aqueous gel of hydroxypropylcellulose (1.00% w/w). pH was adjusted to pH 7.4 by the addition of hydrochloric acid 1M. A heterogeneous suspension is obtained.

### Example 15

Lidocaine free base (2.50% w/w) was added to a hydro-alcoholic gel of hydroxypropylcellulose (1.00% w/w), ethanol (30% w/w) and purified water qs. A clear solution is obtained. Presence of ethanol is herein mandatory to ensure solubilization of lidocaine free base. pH was then further adjusted to pH 7.3 by the addition of hydrochloric acid 1M. A clear, transparent gel is obtained. Viscosity is about 3300 cP.

### Example 16

Lidocaine free base (2.50% w/w) was added to a hydro-alcoholic gel of hydroxypropylcellulose (2.70% w/w), ethanol (30% w/w) and purified water qs. A clear solution is obtained. Presence of ethanol is herein mandatory to ensure solubilization of lidocaine free base. pH was then further adjusted to pH 7.3 by the addition of hydrochloric acid 1M. A clear, transparent gel is obtained. Viscosity is about 46000 cP.

### Example 17

Lidocaine free base (2.50% w/w) was added to a colloidal aqueous dispersion of Carbopol® 974 (1.00% w/w, i.e. same concentration of thickening agent than in Example 15). A homogeneous, transparent gel microscopically free of free drug particles is surprisingly and unexpectedly obtained. pH is about 7.2. Viscosity (BROOKFIELD RV-DVII+ featured with a small sample adapter, spindle S29, 20 rpm, 25° C.) is about 50,000 cP, i.e. a close value of viscosity of gel composition of Example 16.

Through the formation of a complex with a carbomer polymer inventors have found a way to solubilize a poorly water-soluble drug such as lidocaine free base at a pH (7.4 in the present case) at which it would not be soluble otherwise, without the need for either large amounts of pH adjusters, e.g. hydrochloric acid 1M, or organic solvents, e.g. ethanol, which both might be responsible for local skin irritation, dryness, redness and itching.

This gel is then compared to the gel composition of Examples 15 and 16 for in vitro biodistribution of lidocaine into the skin after 24 hours. The relative drug recovery profile of lidocaine after the 24 hour biodistribution is presented in FIG. 5. In FIG. 5, the vertical axis is Drug Recovery as a percent of applied dose, the horizontal axis represents Skin Compartment. The table below summarizes the mean relative absorption data of lidocaine (% of applied drug dose).

| | | | | | | Dermal |
|---|---|---|---|---|---|---|
| | | | | | | to |
| | Total | SC + Epidermal | Dermal | Skin retention | Systemic | Systemic |
| Example | unabsorbed | absorption | absorption | (SC + Epi. + Der.) | absorption | ratio |
| 15 | 68.2 | 3.3 | 1.5 | 4.8 | 3.6 | 0.4 |
| 16 | 66.4 | 4.0 | 2.4 | 6.4 | 3.2 | 0.7 |
| 17 | 66.8 | 4.7 | 1.3 | 6.0 | 1.3 | 1.0 |

Despite lidocaine is involved in the reticulation of carbomer in gel composition of Example 17, lidocaine is not seem to be more retained from this formulation than from other gel compositions. Gel composition of Example 17 does also presents very good drug retention in the skin layers (25% higher than Gel composition of Example 15, and 7% less than Gel composition of Example 16). Further, gel composition of Example 17 presents the lowest systemic absorption (about one third of those of gel composition of Example 15, and about 40% of those of gel composition of Example 16). As a result, gel composition of Example 17 does present the better Dermal:Systemic ratio (1, versus 0.4 for gel composition of Example 15, and versus 0.7 gel composition of Example 16). This is particularly advantageous as the therapeutic target of lidocaine, a local anesthetic, is the nerve ending, which is located in the dermis. Systemic absorption of lidocaine shall be minimized as this may cause fatal adverse events (see “FDA Public Health Advisory Life-Threatening Side Effects with the Use of Skin Products Containing Numbing Ingredients for Cosmetic Procedures”). Noteworthy, one can notice that in vitro skin retention bioavailability of gel composition of Example 17 is the best despite the absence of ethanol, a solvent known to be a very efficient skin permeation enhancer by fluidifying the lipids of the stratum corneum, thereby facilitating the passage of drugs. Absence of ethanol in gel composition of Example 17 would result in improved skin tolerance and patient compliance. This would be further emphasized by the better cosmetic appeal of the carbomer gel carrier, which would be less tacky and sticky than the hydroxypropylcellulose gel composition of Example 16 at a comparable viscosity (about 50,000 cP).

Therefore, inventors have surprisingly found out not only a patient-friendly way to outstandingly solubilize lidocaine in semi-solid alcohol-free vehicles, but also a way to enable skin penetration of ropinirole at levels which would be sufficient for achieving therapeutic concentrations suitable for inducing local anesthesia prior to minor dermal procedures, e.g. skin abrasion, tattooing, skin biopsy or blood sampling.

### Example 18

Compositions as per Example 17 were prepared, varying the drug to carbomer ratio or varying the carbomer type. See Table herein below (percent expressed as percent by weight % w/w).

| Lidocaine | | | | Noveon | Pemulen | | | Viscosity |
|---|---|---|---|---|---|---|---|---|
| free base | C980 | C974 | C971 | AA1 | TR1 | ETD2020 | pH | (cP) |
| 2.5 | 1 | | | | | | 7.3 | 48650 |
| 2.5 | | 1 | | | | | 7.6 | 58600 |
| 2.5 | | | 0.73125 | | | | 7.7 | 7850 |
| 2.5 | | | 0.975 | | | | 7.3 | 9650 |
| 2.5 | | | 1.4625 | | | | 6.1 | 12550 |
| 2.5 | | | 1.95 | | | | 5.6 | 12800 |
| 2.5 | | | 2.5 | | | | 5.1 | 15100 |
| 2.5 | | | 3 | | | | 4.8 | 15650 |
| 2.5 | | | | 1 | | | 7.5 | 40450 |
| 2.5 | | | | | 1 | | 7.4 | 34050 |
| 2.5 | | | | | | 1 | 7.4 | 39950 |

As evidenced by the pH and viscosity values presented herein above, it is possible to control independently pH and viscosity of pharmaceutical compositions containing drug-carbomer complexes. For instance, pH of pharmaceutical compositions containing drug-carbomer complexes can be controlled by changing the drug to carbomer ratio. Viscosity of pharmaceutical compositions containing drug-carbomer complexes can be controlled by selecting an appropriate carbomer type.

### Example 19

Lidocaine free base (2.50% w/w) was added to a colloidal hydro-alcoholic dispersion of Carbopol® 974 (1.00% w/w), ethanol (30% w/w), and water qs. A homogeneous, transparent gel is surprisingly and unexpectedly obtained.

### Example 20

Tetracaine free base (1.00% w/w) was added to a colloidal hydro-alcoholic dispersion of Carbopol® 980 (1.00% w/w), ethanol (49% w/w) and purified water (49% w/w). A macroscopically homogeneous, lightly transparent, fluid gel with a viscosity of about 650 cP is surprisingly and unexpectedly obtained.

### Example 21

Prilocaine free base (1.00% w/w) was added to an aqueous colloidal dispersion of Carbopol® 980 (1.00% w/w). A macroscopically homogeneous, lightly transparent, fluid gel with a viscosity of about 6350 cP is surprisingly and unexpectedly obtained.

### Example 22

Prilocaine free base (1.00% w/w) was added to a colloidal hydro-alcoholic dispersion of Carbopol® 980 (1.00% w/w), ethanol (49% w/w) and purified water (49% w/w). A macroscopically homogeneous, lightly transparent, fluid gel with a viscosity of about 4600 cP is surprisingly and unexpectedly obtained.

### Carbomer Complexes of Anticholinergic Drugs.

### Example 23

Oxybutynin free base (1.00% w/w) was added to a colloidal aqueous dispersion of Carbopol® ETD 2020 (1.00% w/w). A heterogeneous suspension is obtained.

### Example 24

Oxybutynin free base (1.00% w/w) was added to a colloidal hydro-alcoholic dispersion of Carbopol® ETD 2020 (1.00% w/w), ethanol (35% w/w), and water qs. A heterogeneous suspension is obtained.

### Example 25

Oxybutynin free base (1.00% w/w) was added to a colloidal hydro-alcoholic dispersion of Carbopol® ETD 2020 (1.00% w/w), ethanol (37.5% w/w), and water qs. A macroscopically homogeneous, creamy, white emulgel is surprisingly and unexpectedly obtained. Microscopic examination evidences presence of drug crystals.

### Example 26

Oxybutynin free base (1.00% w/w) was added to a colloidal hydro-alcoholic dispersion of Carbopol® ETD 2020 (1.00% w/w), ethanol (40% w/w), and water qs. A macroscopically homogeneous, creamy, white emulgel is surprisingly and unexpectedly obtained. Microscopic examination evidences absence of drug crystals even after 72-hour exposure, i.e. after complete evaporation of the drug carrier. Ethanol is herein mandatory to ensure solubility of the oxybutynin free base-carbomer complex in the media (the least concentration being somewhere between 37.5 and 40.0% w/w).

Interestingly, the gel formulation does present upon evaporation of ethanol some surprising, unexpectedly film-forming ability when applied onto the skin. To the inventors' knowledge, carbomers are not known to exhibit per se such intrinsic film-forming features.

### Example 27

Oxybutynin free base (1.00% w/w) was added to a colloidal hydro-alcoholic dispersion of Carbopol® ETD 2020 (1.00% w/w), ethanol (45% w/w), and water qs. A macroscopically homogeneous, transparent gel is surprisingly and unexpectedly obtained. Microscopic examination evidences absence of drug crystals even after complete evaporation of the drug carrier. U.S. Patent Publications No. US 2005/032441 and US 2005/0064037, the entire content of which is incorporated herein as reference, teach that the only way to obtain oxybutynin carbomer gel formulations is to neutralize the carbomer colloidal dispersions using a base such as diisopropanolamine. Inventors have therefore found a way to manufacture oxybutynin gel with acceptable aesthetic properties (absence of fish-like, strong ammonia smell) against prior art.

Here again, the composition presents film-forming ability.

### Example 28

Oxybutynin free base (1.00% w/w) was added to a colloidal hydro-alcoholic dispersion of Carbopol® ETD 2020 (1.00% w/w), ethanol (50% w/w), and water qs. A macroscopically homogeneous, transparent gel is surprisingly and unexpectedly obtained. Microscopic examination evidences absence of drug crystals even after complete evaporation of the drug carrier.

Here again, though the composition still possess film-forming ability, formation of film is least than those observed in previous Example 27. The formation of the film requires more time, and the strength of the resulting film seems lower. Inventors surmise that this is caused by the larger ethanol amount. Inventors have therefore found that it is surprisingly and unexpectedly possible to produce carbomer film-forming oxybutynin gel formulations by adjusting the ethanol:water ratio so that the oxybutynin carbomer complex is close to its limit of solubilization in said ethanol:water media. Obvious benefits of such film-forming compositions might be water-resistance, and prevention of drug cross-contamination. Film-forming ability vanishes when carbomer are conventionally neutralized with organic amines such as diisopropylamine.

### Example 29

Oxybutynin free base (3.00% w/w) was added to a colloidal hydro-alcoholic dispersion of Carbopol® ETD 2020 (3.00% w/w), ethanol (45% w/w), and water qs. Ratio of drug to carbomer is 1.00, as in previous Example 27. A macroscopically homogeneous, creamy, white emulgel is surprisingly and unexpectedly obtained, while a transparent gel was achieved in Example 27. Inventors surmise that this difference is caused by the larger amount of oxybutynin free base, which requires a higher amount of ethanol to solubilize completely the oxybutynin-carbomer complexes. Microscopic examination evidences absence of drug crystals even after complete evaporation of the drug carrier. Here again, the composition presents film-forming ability.

### Example 30

Oxybutynin free base (3.00% w/w) was added to a colloidal hydro-alcoholic dispersion of Carbopol® ETD 2020 (3.00% w/w), ethanol (50% w/w), and water qs. Ratio of drug to carbomer is 1.00, as in previous Example 27. A macroscopically homogeneous, transparent gel is surprisingly and unexpectedly obtained. Microscopic examination evidences absence of drug crystals even after complete evaporation of the drug carrier.

Here again, the composition presents film-forming ability.

Similar gel compositions have also been achieved, though exhibiting different viscosities, replacing Carbopol® ETD 2020 (3.00% w/w) by short-rheology polymers such as Pemulen® TR1 (3.00% w/w) or Carbopol® 980 (3.00% w/w), or by a long-rheology polymer such as Carbopol® 71 G (3.00% w/w).

### Carbomer Complexes of Antihypertensive Drugs.

### Example 31

Clonidine free base (0.25% w/w) was added to a colloidal aqueous dispersion of Carbopol® 980 (1.00% w/w). A macroscopically homogeneous, lightly opalescent gel is surprisingly and unexpectedly obtained (pH 3.9; viscosity about 25000 cP).

### Example 32

Clonidine free base (0.50% w/w) was added to a colloidal aqueous dispersion of Carbopol® 980 (1.00% w/w). A macroscopically homogeneous, lightly opalescent gel is surprisingly and unexpectedly obtained (pH 4.5; viscosity about 18000 cP).

### Example 33

Clonidine free base (1.00% w/w) was added to a colloidal aqueous dispersion of Carbopol® 980 (1.00% w/w). A macroscopically homogeneous, creamy, white emulgel is surprisingly and unexpectedly obtained (pH 5.5; viscosity about 16500 cP).

### Example 34

Clonidine free base (2.00% w/w) was added to a colloidal aqueous dispersion of Carbopol® 980 (1.00% w/w). A macroscopically homogeneous, transparent gel is surprisingly and unexpectedly obtained (pH 6.8; viscosity about 43000 cP).

### Example 35

Clonidine free base (2.00% w/w) was added to purified water. A heterogeneous suspension was obtained.

### Example 36

Clonidine free base (2.00% w/w) was added to buffer 6.00 (91% w/w) and hydrochloric acid 1M (7% w/w). A clear solution of pH 6.8 (similar to those of composition of Example 34) was obtained.

These examples further evidence the feasibility of solubilizing a poorly water-soluble drug (clonidine free base in the present case) through the formation of complexes with carbomer polymers at pH at which it would not be soluble otherwise, without the need of adding large amounts of skin irritating ingredients, e.g. organic solvents or pH adjusting agents (concentrated HCL 1M in the present case).

### Carbomer Complexes of Benzodiazepines.

### Example 37

Alprazolam (1.00% w/w) was added to a colloidal hydro-alcoholic dispersion of Carbopol® 980 (1.00% w/w), ethanol (49% w/w) and purified water (49% w/w). A macroscopically homogeneous, lightly transparent, fluid gel with a viscosity of about 650 cP is surprisingly and unexpectedly obtained.

### Carbomer Complexes of Anti-Addiction Drugs.

### Example 38

Nicotine free base (4.00% w/w) was added to a colloidal aqueous dispersion of Carbopol® 980 (1.00% w/w). A macroscopically homogeneous, lightly opalescent gel with a viscosity of about 36500 cP is surprisingly and unexpectedly obtained.

### Carbomer Complexes of Analgesic Drugs.

### Example 39

Oxymorphone free base (1.00% w/w) was added to a colloidal aqueous dispersion of Carbopol® 980 (1.00% w/w). A macroscopically homogeneous, creamy white emulgel with a viscosity of about 3350 cP is surprisingly and unexpectedly obtained.

### Carbomer Complexes of Antimetic Drugs.

### Example 40

Granisetron free base (1.00% w/w) was added to a colloidal hydro-alcoholic dispersion of Carbopol® 980 (1.00% w/w), ethanol (49% w/w) and purified water (49% w/w). A macroscopically homogeneous, transparent fluid gel with a viscosity of about 3600 cP is surprisingly and unexpectedly obtained.

### Carbomer Complexes of Neuropathic Pain Drugs.

### Example 41

Gabapentin (1.00% w/w) was added to a colloidal aqueous dispersion of Carbopol® 980 (1.00% w/w). A macroscopically homogeneous, transparent gel with a viscosity of about 5750 cP is surprisingly and unexpectedly obtained.

### Example 42

Gabapentin (1.00% w/w) was added to a colloidal hydro-alcoholic dispersion of Carbopol® 980 (1.00% w/w), ethanol (49% w/w) and purified water (49% w/w). A macroscopically homogeneous, transparent gel with a viscosity of about 33800 cP is surprisingly and unexpectedly obtained.

### Carbomer Complexes of Antialopecia Drugs.

### Example 43

Minoxidil free base (1.00% w/w) was added to a colloidal hydro-alcoholic dispersion of Carbopol® 980 (1.00% w/w), ethanol (49% w/w) and purified water (49% w/w). A macroscopically homogeneous, transparent gel with a viscosity of about 33800 cP is surprisingly and unexpectedly obtained.

In view of the foregoing, it is demonstrated that complexation of drugs with acrylic acid polymers provides a method to enhance solubility and stability of drugs in liquid or semi-solid dosage forms without the need for large amounts of organic solvents and/or pH adjusting acids or alkalis being potentially skin irritating or having an unpleasant odour. Surprisingly, some amine drugs do present the ability to interact with acrylic acid carbomer polymers and to form three-dimensional networks. It has been demonstrated that such interaction between the drug and the polymer prevents or significantly delays drug crystallization. Maintenance of a high thermodynamic activity of the drug within the drug carriers of the present invention has been shown to be responsible for enhanced skin permeation and skin penetration. Further, skin penetration enhancers can be incorporated in the formulations of the present invention. This allows transdermal systemic or local administration of therapeutic levels of drugs, which makes the compositions of the present invention particularly relevant to treat various diseases and conditions. Absence or reduced amounts of potentially skin irritating ingredients makes the compositions of the present invention particularly suitable for transmucosal systemic or local administration of drugs. pH and viscosity of skin-friendly pharmaceutical formulations of the present invention can be controlled independently by varying the ratios of the drug to the polymer, by selecting an appropriate drug form (free base or pharmaceutical salt, thereof), by selecting an appropriate type of carbomer polymer, or by combinations thereof. The present invention further provides an easy way to manufacture carbomer formulations by avoiding the need for the step of neutralization with inorganic alkalis or organic amines.

All patents, publications, and patent applications cited in this specification are herein incorporated by reference as if each individual patent, publication, or patent application was specifically and individually indicated to be incorporated by reference in its entirety for all purposes.

It will be apparent to those skilled in the art that various modifications and variations can be made in the method and composition of the present invention without departing from the spirit or scope of the invention. Thus, it is intended that the present invention include modifications and variations that are within the scope of the appended claims and their equivalents.

What is claimed is:

1. A pharmaceutical composition comprising:
at least one amine drug and an acrylic acid carbomer polymer in the form of a complex that delays crystallization of said at least one amine drug, enhances skin penetration of said at least one amine drug, or allows for the use of no or lower amounts of solvents or pH adjusting agents;
a pharmaceutically acceptable carrier;
optionally at least one non-amine drug
wherein the at least one amine drug uncoils the carboxyl groups of the acrylic acid polymer in the complex so that the viscosity of the composition is not inferior to the viscosity of the same composition not containing the at least one amine drug.

2. The composition of claim 1, wherein the at least one amine drug is selected from the group of primary amines, secondary amines, tertiary amines, aromatic amines, non-aromatic nitrogen-containing heterocyclic amines, azo-amines, imines, and mixtures thereof and is in the form of a free base, a salt thereof, or mixtures thereof.

3. The composition of claim 1 wherein the acrylic acid polymer is a carbomer homopolymer a polycarbophil; a carbomer copolymer; a carbomer interpolymer; or mixtures thereof.

4. The composition of claim 1, wherein the drug and acrylic acid polymer are present in a weight ratio of 10:1 to 1:10.

5. The composition of claim 4, having a pH, a viscosity, or both controlled by varying the form or ratio of the at least one amine drug to the acrylic acid polymer.

6. The composition of claim 1, wherein the pharmaceutically acceptable carrier comprises at least one of at least an alcohol, a glycol, a glycol ether, a glycol ester, an antioxidant, a chelatant, a preservative, a colorant, a fragrance, a flavor, a thickener, a lubricant, a humectant, a moisturizer, a skin emollient, a film-forming agent, a pH adjusting agent, a permeation enhancer, and mixtures thereof.

7. The composition of claim 1 in the form of a occlusive or non-occlusive dosage form selected from the group comprising a solution, a lotion, a gel, a cream, an ointment, an emulsion, a spray, a foam, an aerosol, a patch, and a film.

8. A method of transdermal or transmucosal systemic or local administration of a pharmaceutical composition according to claim 1 to a mammal in need thereof.

9. The method of claim 8, wherein the mammal is a human being.

10. Use of an acrylic acid carbomer polymer to form a complex with at least one amine drug wherein the complex delays crystallization of the at least one amine drug, enhances skin penetration of the at least one amine drug, or allows for the use of no or lower amounts of solvents or pH adjusting agents.

11. Use of at least one amine drug to form a pharmaceutical composition, wherein an acrylic acid carbomer polymer forms a complex with at least one amine drug to delay crystallization of the at least one amine drug, enhance skin penetration of the at least one amine drug, or allows for the use of no or lower amounts of solvents or pH adjusting agents.
